(19) United States
(12) Patent Application Publication
(10) Pub. No.: US 2004/0092591 A1
(43) Pub. Date: May 13, 2004

(54) Therapeutic use of fused bicyclic or tricyclic amino acids

(76) Inventors: David Clive Blakemore, Sandwich (GB); Justin Stephen Bryans, Sandwich (GB); Sophie Caroline Williams, Cambridge (GB)

Correspondence Address: David R. Kurlandsky, Warner-Lambert Company LLC, 2800 Plymouth Road, Ann Arbor, MI, 48105, US

(21) Appl. No.: 10/640,547

(22) Filed: Aug. 13, 2003

Related U.S. Application Data
(60) Provisional application No. 60/421,866, filed on Oct. 28, 2002.

(30) Foreign Application Priority Data
Aug. 15, 2002 (GB) 0219024.7
Oct. 4, 2002 (GB) 0223067.0

(51) Int. Cl. A61K 31/195

(52) U.S. Cl. 514/561

(57) ABSTRACT

The compounds of the instant invention are bicyclic or tricyclic amino acids useful in the treatment of fibromylagia. Pharmaceutical compositions containing one or more of the compounds for use in the treatment of fibromyalgia are also included.

This U.S. Utility application claims the benefit of United Kingdom Application Number 0219024.7 filed Aug. 15, 2002, United Kingdom Application Number 0223067.0 filed Oct. 4, 2002 and U.S. Provisional Application No. 60/421,866 filed Oct. 28, 2002.

### FIELD OF THE INVENTION

This invention relates to the use of novel cyclic amino in the treatment of fibromyalgia.

### BACKGROUND TO THE INVENTION

Gabapentin (Neurontin®) is an anti-convulsant agent that is useful in the treatment of epilepsy and that has recently been shown to be a potential treatment for neurogenic pain. It is 1-(aminomethyl)-cyclohexylacetic acid of structural formula:

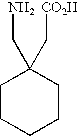

Gabapentin is one of a series of compounds of formula

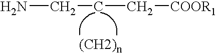

in which R₁ is hydrogen or a lower alkyl radical and n is 4, 5, or 6. These compounds are described U.S. Pat. No. 4,024,175 and its divisional U.S. Pat. No. 4,087,544. Their disclosed uses are: protection against thiosemicarbazide-induced cramp; protection against cardiazole cramp; the cerebral diseases, epilepsy, faintness attacks, hypokinesia, and cranial traumas; and improvement in cerebral functions. The compounds are useful in geriatric patients. The disclosures of the above two patents are hereby incorporated by reference.

WO 99/21824, whose disclosure is also incorporated by reference, discloses further cyclic amino acids that are useful in the treatment of epilepsy, faintness attacks, neurodegenerative disorders, depression, anxiety, panic, pain, neuropathological disorders, gastrointestinal disorders such as irritable bowel syndrome (IBS) and inflammation, especially arthritis. The compounds disclosed include those of the formula:

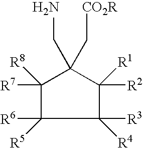

and salts thereof, in which: R is hydrogen or a lower alkyl; and R¹ to R⁸ are each independently selected from hydrogen, straight or branched alkyl of from 1 to 6 carbons, phenyl, benzyl, fluorine, chlorine, bromine, hydroxy, hydroxymethyl, amino, aminomethyl, trifluoromethyl, —CO₂H, —CO₂R¹⁵, —CH₂CO₂H, —CH₂CO₂R¹⁵, —OR¹⁵ wherein R¹⁵ is a straight or branched alkyl of from 1 to 6 carbons, phenyl, or benzyl, R¹ to R⁸ not being simultaneously hydrogen.

International Patent Application Publication No. WO0128978, corresponding to U.S. patent application Ser. No. 60/160725, describes a series of novel bicyclic amino acids, their pharmaceutically acceptable salts, and their prodrugs of formula:

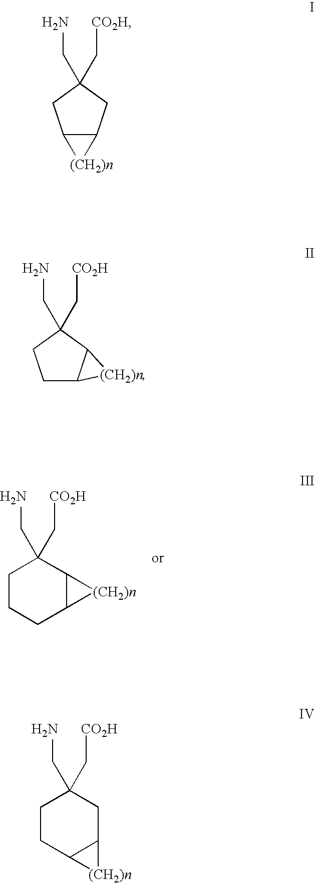

wherein n is an integer of from 1 to 4, where there are stereocentres, each center may be independently R or S, preferred compounds being those of Formulae I-IV above in which n is an integer of from 2 to 4. The compounds are disclosed as being useful in treating a variety of disorders including epilepsy, faintness attacks, hypokinesia, cranial disorders, neurodegenerative disorders, depression, anxiety, panic, pain, neuropathological disorders, and sleep disorders.

Patent application number EP 01400214.1 discloses the use of compounds of formula I to IV above for preventing and treatment of visceral pain, and gastrointestinal disorders.

International Patent Application PCT/IB02/01146, unpublished at the priority date of the present invention, discloses the use of the compounds of the invention of formula (I)-(XXV), below, for a number of disorders. Fibromyalgia is not specifically listed as a suitable utility. The disclosure of PCT/IB02/01146 is incorporated herein in its entirety.

Farrar et al, Pain 94, 149-158 (2001), refers to and uses the data from an unpublished clinical study illustrating the efficacy of a further alpha-2-delta ligand, pregabalin, in the treatment of fibromyalgia.

### SUMMARY OF THE INVENTION

The present invention provides the use of a compound selected from compounds (I)-(XXV), or a pharmaceutically acceptable salt, solvate or pro-drug thereof,

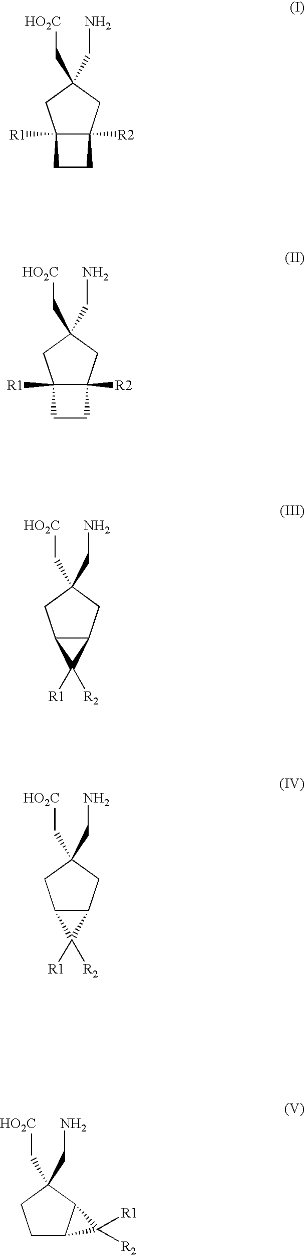

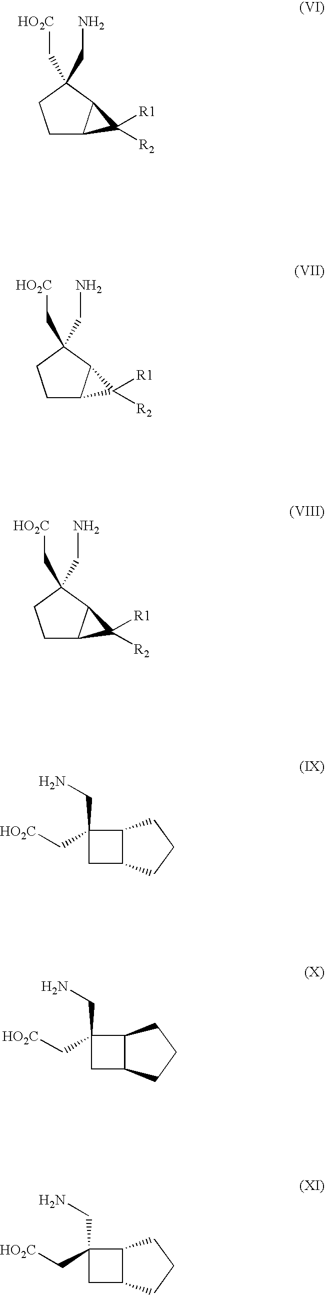

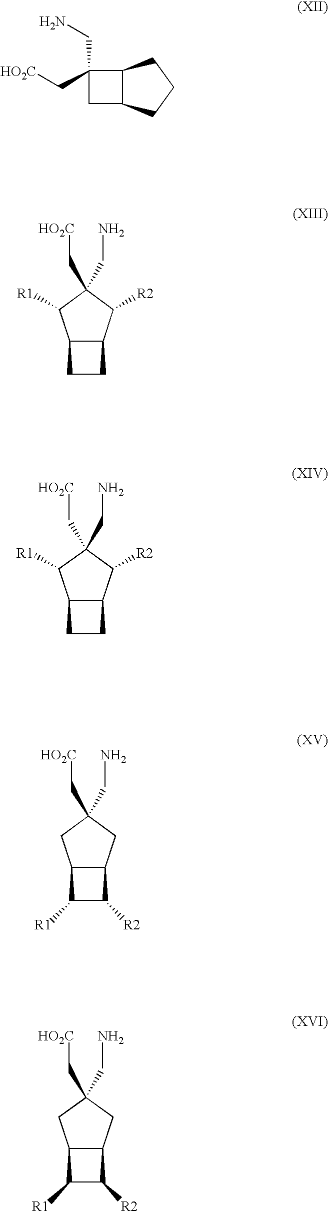

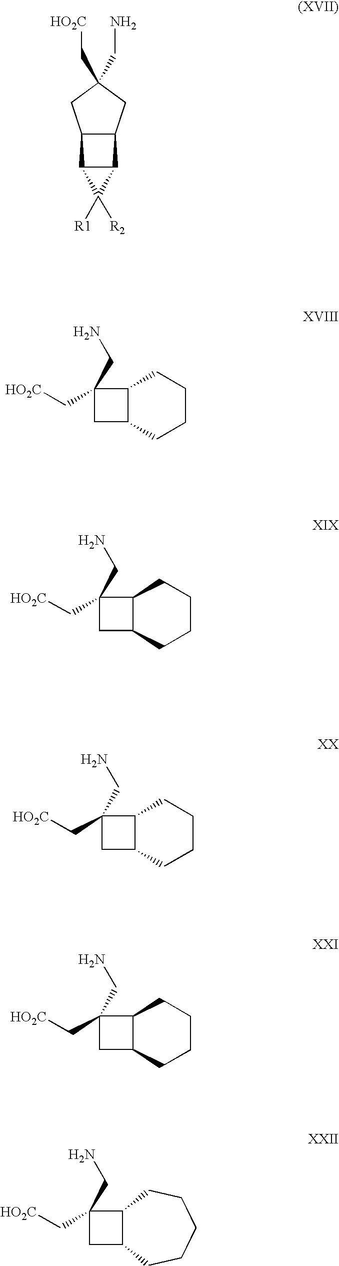

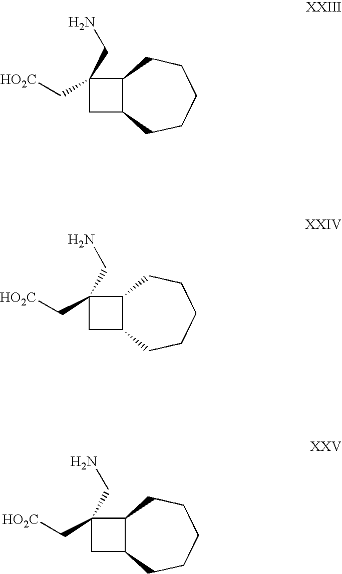

wherein R¹ and R² are each independently selected from H, straight or branched alkyl of 1-6 carbon atoms, cycloalkyl of from 3-6 carbon atoms, phenyl and benzyl, subject to the proviso that, except in the case of a tricyclooctane compound of formula (XVII), R¹ and R² are not simultaneously hydrogen, in the manufacture of a medicament for the treatment of fibromyalgia.

Suitable compounds (including salts, solvates and pro-drugs thereof) are:

((1R,5S)-3-Aminomethyl-1,5-dimethyl-bicyclo[3.2.0]hept-3-yl)-acetic acid;

((1S,5R)-3-Aminomethyl-1,5-dimethyl-bicyclo[3.2.0]hept-3-yl)-acetic acid;

((1R,5S)-3-Aminomethyl-6,6-dimethyl-bicyclo[3.1.0]hex-3-yl)-acetic acid;

((1S,5R)-3-Aminomethyl-6,6-dimethyl-bicyclo[3.1.0]hex-3-yl)-acetic acid;

((1S,2S,5R)-2-Aminomethyl-6,6-dimethyl-bicyclo[3.1.0]hex-2-yl)-acetic acid;

((1R,2S,5S)-2-Aminomethyl-6,6-dimethyl-bicyclo[3.1.0]hex-2-yl)-acetic acid;

((1S,2R,5R)-2-Aminomethyl-6,6-dimethyl-bicyclo[3.1.0]hex-2-yl)-acetic acid;

((1R,2R,5S)-2-Aminomethyl-6,6-dimethyl-bicyclo[3.1.0]hex-2-yl)-acetic acid;

((1R,5R,6S)-6-Aminomethyl-bicyclo[3.2.0]hept-6-yl)-acetic acid;

((1S,5S,6S)-6-Aminomethyl-bicyclo[3.2.0]hept-6-yl)-acetic acid;

((1R,5R,6R)-6-Aminomethyl-bicyclo[3.2.0]hept-6-yl)-acetic acid;

((1S,5S,6R)-6-Aminomethyl-bicyclo[3.2.0]hept-6-yl)-acetic acid;

cis-((1S,2R,4S,5R)-3-Aminomethyl-2,4-dimethyl-bicyclo[3.2.0]hept-3-yl)acetic acid;

trans-((1S,2R,4S,5R)-3-Aminomethyl-2,4-dimethyl-bicyclo[3.2.0]hept-3yl)-acetic acid;

((1S,5R,6S,7R)-3-Aminomethyl-6,7-dimethyl-bicyclo[3.2.0]hept-3-yl)-acetic acid;

((1S,5R,6R,7S)-3-Aminomethyl-6,7-dimethyl-bicyclo[3.2.0]hept-3-yl)-acetic acid;

((1R,2S,5S)-7-Aminomethyl-3,3-dimethyl-tricyclo[3.3.0.0]oct-7-yl)-acetic acid;

((1R,6R,7S)-7-Aminomethyl-bicyclo[4.2.0]oct-7-yl)-acetic acid;

((1S,6S,7S)-7-Aminomethyl-bicyclo[4.2.0]oct-7-yl)-acetic acid;

((1R,6R,7R)-7-Aminomethyl-bicyclo[4.2.0]oct-7-yl)-acetic acid;

((1S,6S,7R)-7-Aminomethyl-bicyclo[4.2.0]oct-7-yl)-acetic acid;

((1R,7R,8S)-8-Aminomethyl-bicyclo[5.2.0]non-8-yl)-acetic acid;

((1S,7S,8S)-8-Aminomethyl-bicyclo[5.2.0]non-8-yl)-acetic acid;

((1R,7R,8R)-8-Aminomethyl-bicyclo[5.2.0]non-8-yl)-acetic acid; and

((1S,7S,8R)-8-Aminomethyl-bicyclo[5.2.0]non-8-yl)-acetic acid.

Preferred compounds (including salts, solvates and pro-drugs thereof) are:

[(1R,5R,6S)-6-(Aminomethyl)bicyclo[3.2.0]hept-6-yl]acetic acid;

[(1S,5S,6R)-6-(Aminomethyl)bicyclo[3.2.0]hept-6-yl]acetic acid;

[(1RS,5RS,6RS)-6-(Aminomethyl)bicyclo[3.2.0]hept-6-yl]acetic acid;

[(1RS,6RS,7SR)-7-(Aminomethyl)bicyclo[4.2.0]oct-7-yl]acetic acid; and

[(1RS,6RS,7RS)-7-(Aminomethyl)bicyclo[4.2.0]oct-7-yl]acetic acid.

A particularly preferred compound (including salts, solvates and pro-drugs thereof) is [(1R,5R,6S)-6-(Aminomethyl)bicyclo[3.2.0]hept-6-yl]acetic acid.

The present compounds can exist in unsolvated forms as well as solvated forms, including hydrated forms. In general, the solvated forms, including hydrated forms, which may contain isotopic substitutions (e.g. D20, d6-acetone, d6-DMSO), are equivalent to unsolvated forms and are encompassed within the scope of the present invention.

Certain of the compounds of the present invention possess one or more chiral centers and each center may exist in the R(D) or S(L) configuration. The present invention includes all enantiomeric and epimeric forms as well as the appropriate mixtures thereof. Separation of diastereoisomers or cis and trans isomers may be achieved by conventional techniques, e.g. by fractional crystallisation, chromatography or H.P.L.C. of a stereoisomeric mixture of a compound of the invention or a suitable salt or derivative thereof. An individual enantiomer of a compound of the invention may also be prepared from a corresponding optically pure intermediate or by resolution, such as by H.P.L.C. of the corresponding racemate using a suitable chiral support or by fractional crystallisation of the diastereoisomeric salts formed by reaction of the corresponding racemate with a suitable optically active acid or base, as appropriate.

Since amino acids are amphoteric, pharmacologically compatible salts can be salts of appropriate non-toxic inorganic or organic acids or bases. Suitable acid addition salts are the hydrochloride/chloride, hydrobromide/bromide, hydroiodide/iodide, sulphate, bisulphate, nitrate, phosphate, hydrogen phosphate, acetate, fumarate, aspartate, besylate, bicarbonate/carbonate, camsylate, D and L-lactate, D and L-tartrate, edisylate, mesylate, malonate, orotate, gluceptate, methylsulphate, stearate, glucuronate, 2-napsylate, tosylate, hibenzate, nicotinate, isethionate, malate, maleate, citrate, gluconate, succinate, saccharate, benzoate, esylate, and pamoate salts. Suitable base salts are formed from bases which form non-toxic salts and examples are the sodium, potassium, aluminium, calcium, magnesium, zinc, choline, diolamine, olamine, arginine, glycine, tromethamine, benzathine, lysine, meglumine and diethylamine salts. Salts with quaternary ammonium ions can also be prepared with, for example, the tetramethylammonium ion. The compounds of the invention may also be formed as a zwitterion.

A suitable salt of compounds of the present invention is the hydrochloride salt. For a review on suitable salts see Berge et al, J. Pharm. Sci., 66, 1-19, 1977.

Also included within the present scope of the compounds of the invention are polymorphs thereof.

Prodrugs of the above compounds are included in the scope of the instant invention. The effectiveness of an orally administered drug is dependent upon the drug's efficient transport across the mucosal epithelium and its stability in entero-hepatic circulation. Drugs that are effective after parenteral administration but less effective orally, or whose plasma half-life is considered too short, may be chemically modified into a prodrug form. A prodrug is a drug which has been chemically modified and may be biologically inactive at its site of action, but which may be degraded or modified by one or more enzymatic or other in vivo processes to the parent bioactive form. This chemically modified drug, or prodrug, should have a different pharmacokinetic profile to the parent, enabling easier absorption across the mucosal epithelium, better salt formulation and/or solubility, improved systemic stability (for an increase in plasma half-life, for example). These chemical modifications may be

(1) Ester or amide derivatives which may be cleaved by, for example, esterases or lipases. For ester derivatives, the ester is derived from the carboxylic acid moiety of the drug molecule by known means. For amide derivatives, the amide may be derived from the carboxylic acid moiety or the amine moiety of the drug molecule by known means.

(2) Peptides which may be recognized by specific or nonspecific proteinases. A peptide may be coupled to the drug molecule via amide bond formation with the amine or carboxylic acid moiety of the drug molecule by known means.

(3) Derivatives that accumulate at a site of action through membrane selection of a prodrug form or modified prodrug form.

(4) Any combination of 1 to 3.

It will further be appreciated by those skilled in the art that certain moieties known to those skilled in the art as “pro-moieties”, for example as described in “Design of Prodrugs” by H Bundgaard (Elsevier) 1985, may be placed on appropriate functionalities when such functionalities are present in compounds of the invention also to form a “prodrug”. Further, certain compounds of the invention may act as prodrugs of other compounds of the invention. All protected derivatives, and prodrugs, of the compounds of the invention are included within the scope of the invention.

Research has shown that the oral absorption of certain drugs may be increased by the preparation of “soft” quaternary salts. The quaternary salt is termed a “soft” quaternary salt since, unlike normal quaternary salts, e.g., R—N⁺(CH₃)₃, it can release the active drug on hydrolysis. “Soft” quaternary salts have useful physical properties compared with the basic drug or its salts. Water solubility may be increased compared with other salts, such as the hydrochloride, but more important there may be an increased absorption of the drug from the intestine. Increased absorption is probably due to the fact that the “soft” quaternary salt has surfactant properties and is capable of forming micelles and unionized ion pairs with bile acids, etc., which are able to penetrate the intestinal epithelium more effectively. The prodrug, after absorption, is rapidly hydrolyzed with release of the active parent drug.

Aminoacyl-glycolic and -lactic esters are known as prodrugs of amino acids (Wermuth C. G., Chemistry and Industry, 1980:433-435). The carbonyl group of the amino acids can be esterified by known means. Prodrugs and soft drugs are known in the art (Palomino E., Drugs of the Future, 1990; 15(4):361-368). The last two citations are hereby incorporated by reference.

The biological activity of the compounds of the invention may be measured in a radioligand binding assay using [³H]gabapentin and the α₂δ subunit derived from porcine brain tissue (Gee N. S., Brown J. P., Dissanayake V. U. K., Offord J., Thurlow R., Woodruff G. N., ADVANCEADVANCE J. Biol. Chem., 1996;271:5879-5776). Results may be expressed in terms of μM or nM α₂δ binding affinity.

The therapeutic compounds can be administered, for example but not limited to the following route: orally, buccally or sublingually in the form of tablets, capsules, multi-and nano-particulates, gels, films (incl. muco-adhesive), powder, ovules, elixirs, lozenges (inc. liquid-filled), chews, solutions, suspensions and sprays. The compounds of the invention may also be administered as osmotic dosage form, or in the form of a high energy dispersion or as coated particles or fast-dissolving, fast -disintegrating dosage form as described in Ashley Publications, 2001 by Liang and Chen.

The therapeutic compounds can also be administered by injection, that is, intravenously, intramuscularly, intracutaneously, intraduodenally, or intraperitoneally, intraarterially, intrathecally, intraventricularly, intraurethrally, intrasternally, intracranially, intraspinally or subcutaneously, or they may be administered by infusion, needle-free injectors or implant injection techniques.

Also, the therapeutic compounds can be administered intranasally or by inhalation.

Alternatively, the therapeutic compounds may be administered topically to the skin, mucosa, dermally or transdermally, for example, in the form of a gel, hydrogel, lotion, solution, cream, ointment, dusting powder, dressing, foam, film, skin patch, wafers, implant, sponges, fibres, bandage, microemulsions and combinations thereof.

Alternatively, the therapeutic compounds can be administered rectally, for example in the form of a suppository or pessary. They may also be administered by vaginal route.

The therapeutic compounds may also be administered by the ocular route. They may also be administered in the ear, using for example but not limited to the drops.

The therapeutic compounds may also be used in combination with a cyclodextrin. Alpha-, beta- and gamma-cyclodextrins are most commonly used and suitable examples are described in WO-A-91/11172, WO-A-94/02518 and WO-A-98/55148.

The term ‘administered’ includes delivery by viral or non-viral techniques. Viral delivery mechanisms include but are not limited to adenoviral vectors, adeno- associated viral (AAV) vectors, herpes viral vectors, retroviral vectors, lentiviral vectors, and baculoviral vectors. Non-viral delivery mechanisms include lipid mediated transfection, lipsomes, immunoliposomes, lipofectin, cationic facial amphiphiles (CFAs) and combinations thereof. The routes for such delivery mechanisms include but are not limited to mucosal, nasal, oral, parenteral, gastrointestinal, topical or sublingual routes.

The pharmaceutical preparation of the therapeutic compounds is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsules, tablet, cachet, or lozenge itself, or it can be the appropriate number of any of these in packaged form. The quantity of active component in a unit dose preparation may be varied or adjusted from 0.1 mg to 1 g according to the particular application and the potency of the active component. In medical use the drug may be administered three times daily as, for example, capsules of 100 or 300 mg. In therapeutic use, the compounds utilized in the pharmaceutical method of this invention are administered at the initial dosage of about 0.01 mg to about 100 mg/kg daily. A daily dose range of about 0.01 mg to about 100 mg/kg is preferred. The dosages, however, may be varied depending upon the requirements of the patient, the severity of the condition being treated, and the compound being employed. Determination of the proper dosage for a particular situation is within the skill of the art. Generally, treatment is initiated with smaller dosages which are less than the optimum dose of the compound. Thereafter, the dosage is increased by small increments until the optimum effect under the circumstances is reached. For convenience, the total daily dosage may be divided and administered in portions during the day, if desired.

The pharmaceutical composition according to the present invention can, if desired, also contain one or more other compatible therapeutic agents. In particular, the composition can be combined with any one or more compounds useful in the treatment of pain, such as those listed above. Thus, the present invention presents a pharmaceutical composition comprising a compound selected from formula (I)-(XXV), one or more other pharmacologically active agents and one or more pharmaceutically acceptable carriers.

### General Methods

The above compounds can be synthesised from the ketones (1)-(12) below, in which R¹ and R² have the same meanings as give above:

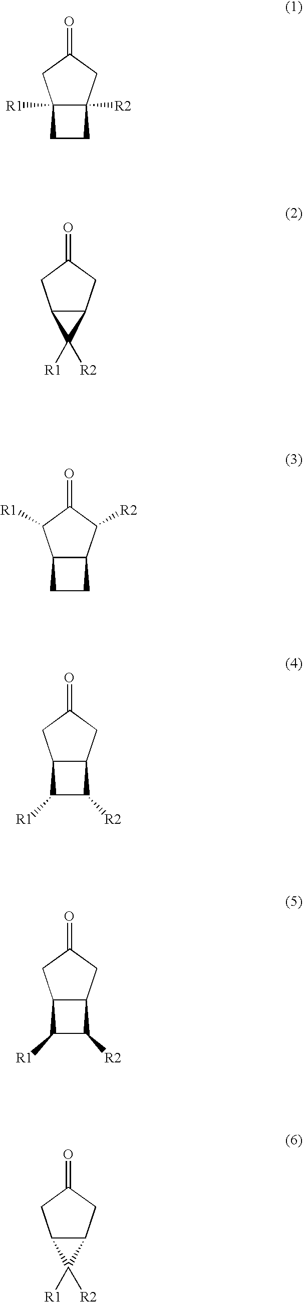

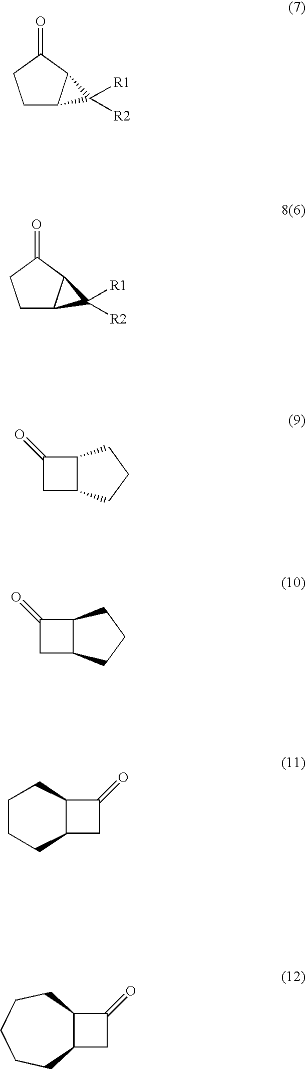

Intermediates of formulae (1) to (6) above are believed to be novel and constitute a further aspect of the present invention. Particularly suitable intermediate ketones according to the present invention are selected from:

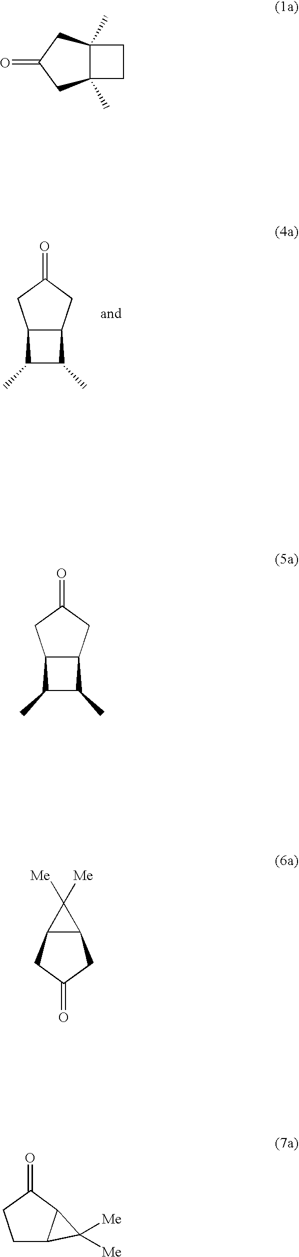

Various methods for synthesizing the above ketones are set out below:

### A. Syntheses of Ketones 1-12

### (1) Synthesis of Ketones of Type (1)

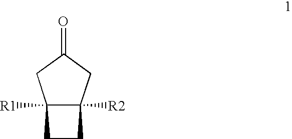

For Example:

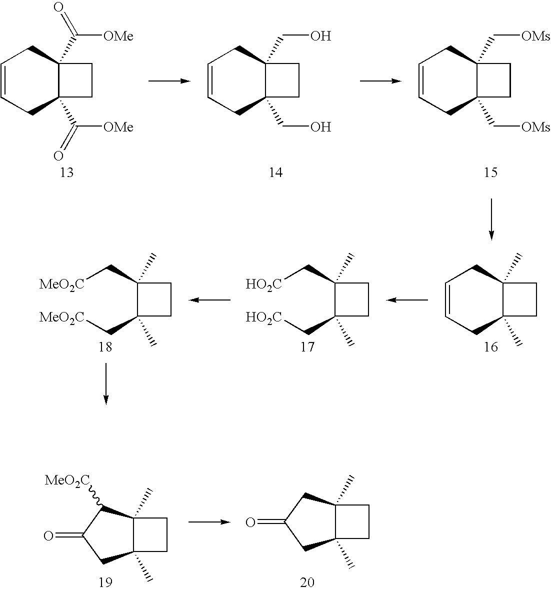

(a) The known diester (13) is reduced to diol (14) e.g. by lithium aluminium hydride in an organic solvent e.g. tetrahydrofuran or diethyl ether at a temperature of 0° C. to reflux.

(b) The diol (14) is added to methylsulfonyl chloride in pyridine or triethylamine in dichloromethane a −60° C. to 40° C. to produce a dimesylate of formula (15).

(c) The dimesylate (15) is added to a solution of lithium aluminium hydride in a solvent such as tetrahydrofuran or diethyl ether at a temperature of from 0° C. to reflux to produce an alkene of formula (16).

(d) The alkene (16) above is added

to a mixture of carbon tetrachloride or ethyl acetate and acetonitrile to which water, sodium periodate and ruthenium (III) chloride were added, and stirred at a temperature from −40° C. to 80° C. to produce carboxylic acid of formula (17); or

to a mixture of potassium permanganate in water and dichloromethane in the presence of a phase transfer catalyst such as tetrabutylammonium bromide to produce (17).

(e) The carboxylic acid (17) is added to a mixture of an alcohol such as methanol and a concentrated acid such as sulphuric acid or hydrochloric acid at a temperature of room temperature to reflux to produce diester of formula (18).

(f) The diester (18) above is added to a strong base such as sodium hydride or potassium tert-butoxide in a solvent such as tetrahydrofuran at reflux temperature to give ketone (19).

(g) The ketone (19) above is added to a mixture of dimethyl sulphoxide and water at a temperature of 100-180° C. to produce ketone of formula (20).

### (2) Synthesis of Ketones of Type (4) and (5)

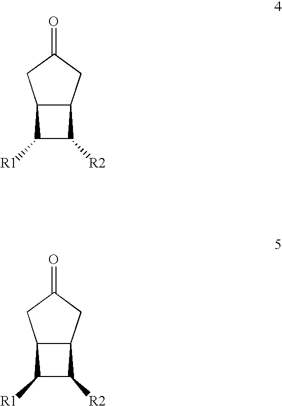

For Example:

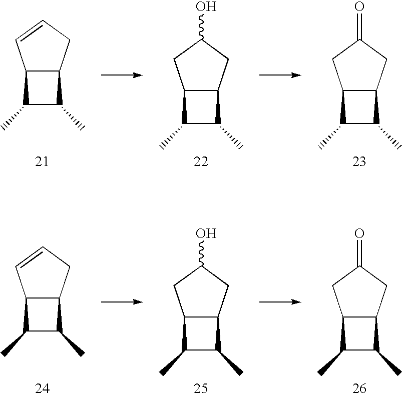

(a) The known alkene (21), see B. D. Kramer, P. D. Bartlett, J. Am. Chem. Soc., 1972, 94, 3934, is mixed with an organoborane such as disiamylborane, thexylborane or 9-BBN in a solvent such as diethyl ether or tetrahydrofuran at a temperature of 0° C. to room temperature. The resulting organoborane is mixed with a solution of concentrated sodium hydroxide and hydrogen peroxide to give an alcohol of formula (22).

(b) The alcohol (22) is oxidized, e.g. with an oxidising agent such as chromium trioxide, pyridinium dichromate or pyridinium chlorochromate in a solvent such as dichloromethane or acetone to give the ketone of formula (23).

A similar process can be used for ketone (25) except that the starting material is the known alkene (24), see B. D. Kramer, P. D. Bartlett, supra.

### (3). Synthesis of Ketones of Type (3)

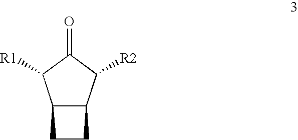

For Example:

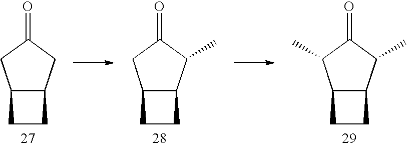

(a) The known ketone (27) see patent application Ser. No. 60/160725, is added to a strong base such as lithium diisopropylamide or lithium hexamethyldisilazide followed by a methylating agent such as methyl iodide in a solvent such as tetrahydrofuran or diethyl ether at a temperature of between −100° C. and room temperature to give the ketone of formula (28).

(b) The ketone of formula (46) above is further methylated with a methylating agent such as methyl iodide in the presence of a strong base such as lithium diisopropylamide or lithium hexamethyldisilazide in a solvent such as tetrahydrofuran or diethyl ether at a temperature of between −100° C. and room temperature to give the product ketone of formula (29).

### (4). Synthesis of Ketones of Type (9) and (10)

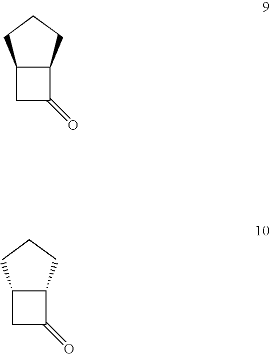

These ketones are known compounds, see L. Y. Chen, L. Ghosez, Tetrahedron Letters, 1990, 31, 4467; C. Houge, A. M. Frisque-Hesbain, A. Mockel, L. Ghosez, J. P. Declercq, G. Germain, M. Van Meerssche, J. Am. Chem. Soc., 1982, 104, 2920.

These ketones may also be prepared from the known unsaturated ketone of general formula (76)

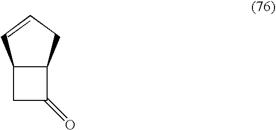

by reduction by hydrogenation with a suitable catalyst such as Pd/C in a suitable solvent such as ethyl acetate.

### (5). Synthesis of Ketones of Type (2)

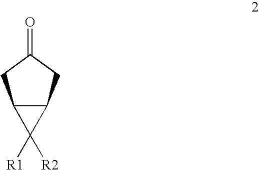

For Example:

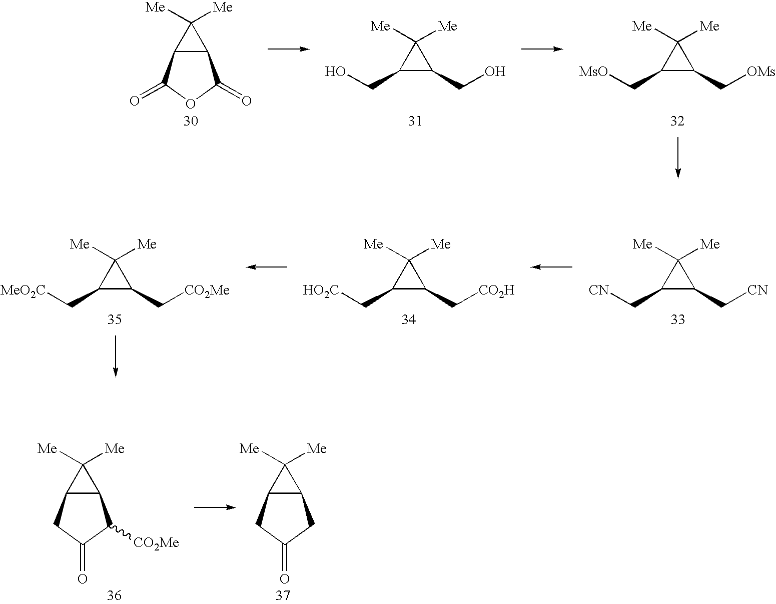

(a) The known carbamate (30), see W. Von der Saal, R. Reinhardt, H. M. Seidenspinner, J. Stawitz, H. Quast, Liebigs Ann. Chem., 1989, 703; Z. Cekovic, R. Matovic, J. Serb. Chem. Soc., 1988, 53, 595, is reduced using lithium aluminium hydride in a solvent such as tetrahydrofuran or diethyl ether at a temperature of 0° C. to reflux to give diol (31).

(b) The diol (31) is added to methylsulphonyl chloride in pyridine or triethylamine in dichloromethane at a temperature of −60° C. to 40° C. to produce dimesylate of formula (32).

(c) The dimesylate (32) is added to sodium or potassium cyanide in a solvent such as tetrahydrofuran, diethyl ether, dimethylsulphoxide or dimethylformamide at a temperature of 0° C. to reflux to give the dicyanide of structure (33).

(d) The dicyanide (33) is added to a concentrated solution of potassium or sodium hydroxide at a temperature of 50° C. to reflux to give diacid (34).

(e) The diacid (34) is esterified to diester (35) by addition:

to a mixture of iodomethane in a solvent selected from dichloromethane, chloroform, tetrahydrofuran, toluene or 1,4-dioxane to which a base such as 1,8-diazabicyclo[5.4.0]undec-7-ene (DBU), triethylamine or 1,5-diazabicyclo[4.3.0]non-5-ene (DBN) is added and stirred at a temperature from −40° C. to 110° C.; or

to a mixture of methanol and a concentrated acid such as sulphuric acid or hydrochloric acid at a temperature ranging from 0° C. to 100° C.; or

to trimethylsilyldiazomethane and methanol in benzene or toluene at a temperature from −40° C. to 100° C.; or

to diazomethane in a solvent such as benzene, toluene, dichloromethane at a temperature from −40° C. to 40° C.

(f) The diester (35) is added to a strong base such as sodium hydride or potassium tert-butoxide in a solvent such as tetrahydrofuran at reflux temperature to give ketone (36).

(g) The ketone (36) above is added to a mixture of dimethyl sulphoxide and water at a temperature of 100-180° C. to produce ketone of formula (37).

### (6). Synthesis of Ketones of Type 7 and 8

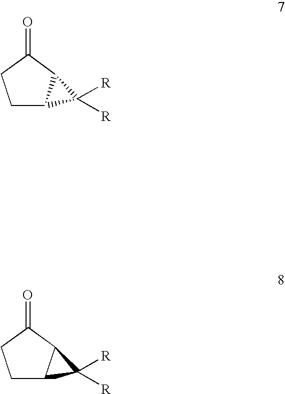

Ketones of this type can be made using ruthenium complexes, see S -W. Park, J -H. Son, S -G. Kim, K. H. Ahn, Tetrahedron: Asymmetry, 1999, 10, 1903.

For Example:

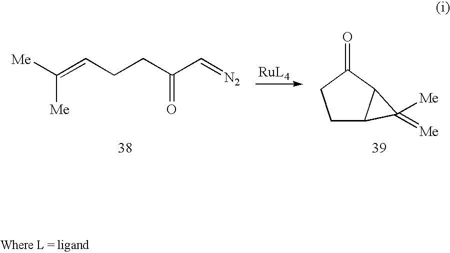

The known alkene (38), see H. Nishiyama, Y. Itoh, H. Matsumoto, S. B. Park, K. Itoh, J. Am. Chem. Soc., 1994, 116, 2223, was stirred with a ruthenium catalyst such as Cl₂Ru(pybox-ip)(CH₂═CH₂) in a solvent such as dichloromethane or chloroform at a temperature of 0° C. to room temperature to give ketone of structure (39).

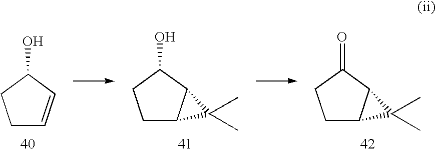

(a) The known alcohol (40), see M. Asami, Bull. Chem. Soc. Jpn., 1990, 63, 721; T. Sato, Y. Gotoh, Y. Wakabayashi, T. Fujisawa, Tetrahedron Letters, 1983, 24, 4123, is mixed with diiodomethane and an alkylzinc such as dimethylzinc or diethylzinc or a zinc-copper couple in a solvent such as toluene or benzene at a temperature of −60° C. to reflux to give an alcohol of formula (41).

(b) The alcohol of formula (41) is added to an oxidising agent such as chromium trioxide, pyridinium dichromate or pyridinium chlorochromate in a solvent such as dichloromethane or acetone to give the ketone of formula (42).

### (7). Synthesis of Ketones of Type (6)

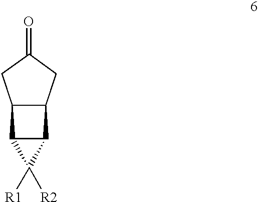

For Example:

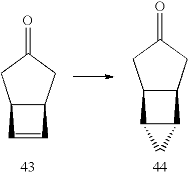

The known ketone (43), see W. A. Wilczak, D. I. Schuster, Tetrahedron Letters, 1986, 27, 5331; D. I. Schuster, J. Eriksen, J. Org. Chem, 1979, 44, 4254, is mixed with diiodomethane and an alkylzinc such as dimethylzinc or diethylzinc or a zinc-copper couple in a solvent such as toluene or benzene at a temperature of 60° C. to reflux to give ketone of structure (44).

### (8). Synthesis of Ketones of Type (11) and (12)

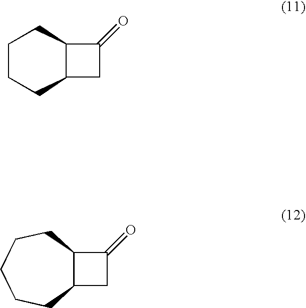

Preparation of ( 1) can be found in the following references:

Ogino, Toshio. Preparation of bicyclo[4.2.0]octan-7-ones. Niigata Daigaku Kyoikugakubu Kiyo, Shizen Kagaku Hen (1973), 15 26-33.

Marko, Istvan; Ronsmans, Bruno; Hesbain-Frisque, Anne Marie; Dumas, Stephane; Ghosez, Leon; Ernst, Beat; Greuter, Hans. Intramolecular [2+2] cycloadditions of ketenes and keteniminium salts to olefins. J. Am. Chem. Soc. (1985), 107(7), 2192-4.

Chen, Lian Yong; Ghosez, Leon. Study of chiral auxiliaries for the intramolecular [2+2] cycloaddition of a keteniminium salt to an olefinic double bond. A new asymmetric synthesis of cyclobutanones. Tetrahedron Lett. (1990), 31(31), 4467-70.

Preparation of (12) can be found in Marko et al., supra.

### B. Conversion of Ketone Starting Materials into Amino Acids of the Invention

The above ketones can be transformed into amino acids using one of the following general methods A to E, as illustrated below for ketone (1) where R¹═R²=methyl.

Method A:

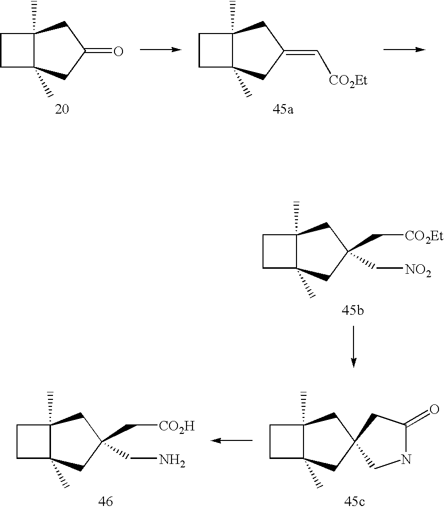

(a) The ketone (20) is converted to unsaturated ester (45a) by reaction with with a trialkylphosphonoacetate such as triethylphosphonoacetate in the presence of a base. Suitable bases include sodium hydride, potassium hydride, lithium- or sodium- or potassium-hexamethyldisilazide, butyllithium or potassium tert-butoxide. The reaction may be carried out in a polar aprotic organic solvent such as tetrahydrofuran, dimethylformamide, diethyl ether or dimethylsulfoxide at a temperature in the range from −78° C. to 100° C.

(b) Nitromethane is added to the unsaturated ester (45a) by a Michael addition reaction in the presence of a base and in a polar aprotic organic solvent at a temperature of −20° C. to 100° C. to give the nitroester (45b). Suitable bases include tetrabutylammonium fluoride, tetramethylguanidine, 1,5-diaza-bicyclo[4,3,0]non-5-ene, 1,8-diazabicyclo[5,4,0]undec-7-ene, a sodium or potassium alkoxide such as potassium tert-butoxide, potassium carbonate, sodium hydride or potassium fluoride. Suitable organic solvents include tetrahydrofuran, diethyl ether, dimethylformamide, dimethylsulphoxide, benzene, toluene, dichloromethane, chloroform or tetrachloromethane.

(c) Reduction of the nitro ester (45b) and ring closure by reaction of the resulting amino group with the ester group gives the cyclic lactam (45c). Hydrogenation may be in the presence of a catalyst such as Raney nickel, palladium on charcoal or rhodium catalyst or other nickel or palladium containing catalyst in a solvent such as methanol, ethanol, isopropanol, ethyl acetate, acetic acid, 1,4-dioxane, chloroform or diethyl ether at a temperature in the range from 20° C. to 80° C.

(d) Hydrolysis of the cyclic lactam (45c) e.g. using aqueous hydrochloric acid at a concentration of from 0.01 M to 12 M and optionally in the presence of a solvent such as 1,4-dioxane, acetic acid or water produces the amino acid (46).

Method B:

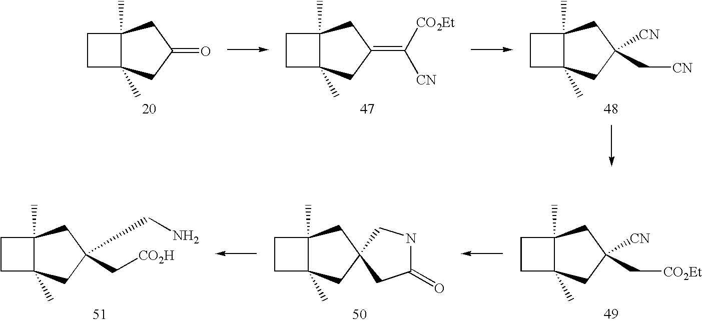

(a) The ketone (20) is condensed with an alkyl cyanoacetate, for example ethyl cyanoacetate in an organic solvent selected from toluene, benzene, xylenes or n-heptane to which acetic acid and β-alanine or ammonium acetate, or piperidine are added. The mixture is stirred at a temperature from 0° C. to 150° C. with removal of water by, for example, use of a Dean-Stark trap or activated molecular sieves, to produce the cyanoester of formula (47).

(b) The cyanoester (47) is converted to dicyanide (48) by treatment with potassium cyanide or sodium cyanide in water and ethanol or methanol. The mixture is refluxed and water is removed by, for example, use of a Dean-Stark trap.

(c) The cyanomethyl group of dicyanide (48) converted to an ethoxycarbonylmethyl group by reaction with ethanol in toluene or benzene saturated with gaseous hydrochloric acid. The reaction temperature may be from −30° C. to 40° C.

(d) The cyano-group of the resulting cyanoester (49) is reduced by hydrogenation in methanol, ethanol or ethyl acetate using a catalyst such as nickel, palladium, platinum or rhodium at a temperature from 15° C. to 60° C., after which ring closure gives lactam (50).

(e) Hydrolysis of the lactam (50) e.g. using aqueous hydrochloric acid at a concentration of from 0.01 M to 12 M and optionally in the presence of a solvent such as 1,4-dioxane, acetic acid or water produce the amino acid (51).

Method C:

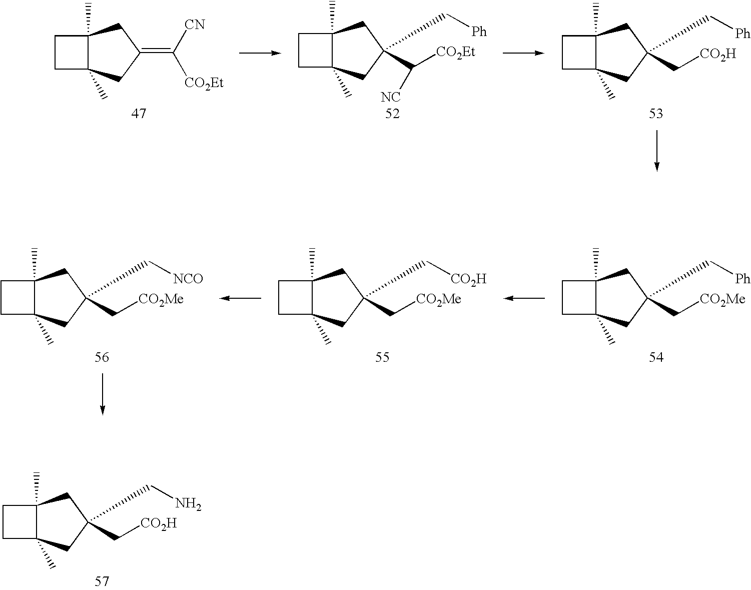

(a) Cyanoester (47) is added to a mixture of benzylmagnesium chloride, bromide or iodide, in a dry solvent e.g. tetrahydrofuran, 1,4-dioxane, n-heptane, toluene, diethyl ether, or tert-butyl methyl ether at a temperature from −100° C. to 110° C. resulting in cyanoester of formula (52).

(b) The cyano group of cyanoester (52) is removed by means of a base e.g. potassium hydroxide, sodium hydroxide, lithium hydroxide or cesium hydroxide in a solvent e.g. ethylene glycol, 2-methoxyethyl ether, 1,4-dioxane or diethylene glycol. The mixture is stirred at a temperature from 25° C. to 250° C. to produce the carboxylic acid of formula (53).

(c) The carboxylic acid group of acid (53) is protected by conversion to its alkyl of 1-6 carbon atoms ester, e.g. its methyl ester (54). For this purpose, acid (53) may be added

to a mixture of iodomethane in a solvent selected from dichloromethane, chloroform, tetrahydrofuran, toluene or 1,4-dioxane to which a base such as 1,8-diazabicyclo[5.4.0]undec-7-ene (DBU), triethylamine or 1,5-diazabicyclo[4.3.0]non-5-ene (DBN) is added and stirred at a temperature from −40° C. to 110° C.; or

to a mixture of methanol and a concentrated acid such as sulphuric acid or hydrochloric acid at a temperature ranging from 0° C. to 100° C.; or

to trimethylsilyldiazomethane and methanol in benzene or toluene at a temperature from −40° C. to 100° C.; or

to diazomethane in a solvent such as benzene, toluene, dichloromethane at a temperature from −40° C. to 40° C.

(d) The phenyl group of the resulting ester (54) is oxidized to a carboxylic acid group by treatment with sodium periodate and ruthenium (III) chloride in a mixture of carbon tetrachloride or ethyl acetate and acetonitrile to which water is added. The mixture is stirred at a temperature from −40° C. to 80° C. to give carboxylic acid (55).

(e) The carboxylic acid group of acid (55) is converted to isocyanate by addition

to a mixture of a base selected from triethylamine or diisopropylethylamine and a solvent selected from toluene, benzene, xylenes, tetrahydrofuran, diethyl ether or n-heptane to which diphenylphosphoryl azide (DPPA) is added and stirring at a temperature from 0° C. to 150° C. to produce the isocyanate of formula (26); or

to ethyl chloroformate or isobutyl chloroformate and a base such as triethylamine or diisopropylethylamine in tetrahydrofuran or acetone or diethyl ether at a temperature of −40° C. to 78° C. followed by addition of sodium azide in water and tetrahydrofuran or acetone followed by addition of toluene or benzene and refluxing.

(f) The isocyanate and ester groups of compound (56) are simultaneously hydrolysed to amino and carboxylic acid groups, e.g. by aqueous hydrochloric acid at a concentration of from 0.01 M to 12 M optionally in the presence of a solvent such as 1,4-dioxane, acetic acid or water to produce the amino acid (57).

Method D:

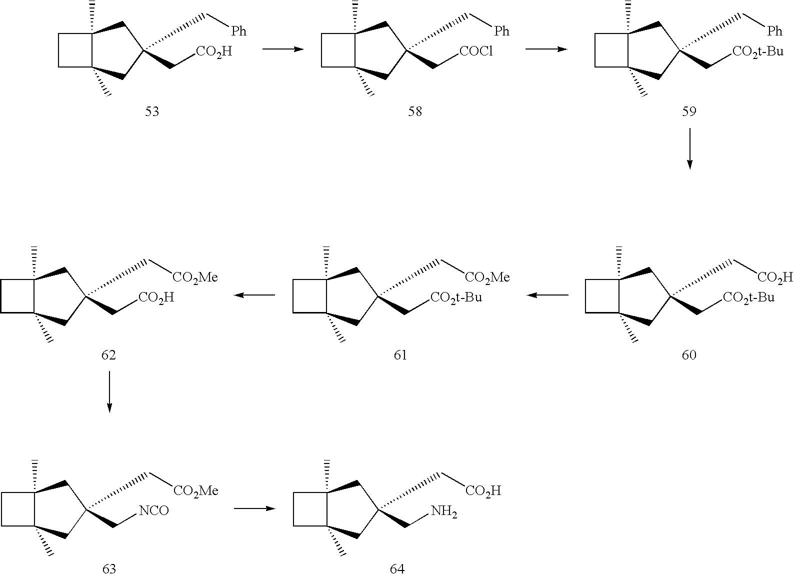

(a) As a first stage in protecting the carboxylic acid group of acid (53), it is converted to its chloride (58) by reaction at a temperature of from −40° C. to 110° C. with e.g. oxalyl chloride or thionyl chloride in an aprotic organic solvent e.g dichloromethane, chloroform, diethyl ether, toluene or tert-butyl methyl ether to which 0.01 mol percent to 10 mol percent of N,N-dimethylformamide (DMF) is added.

(b) The chloride (58) is converted to its tert-butyl ester, e.g. by reaction with tert-butyl alcohol in an aprotic organic solvent e.g. dichloromethane, chloroform, diethyl ether, toluene, or tert-butyl methyl ether to which N,N-diisopropylethylamine (DIPEA) or triethylamine is added. The reaction mixture is stirred at a temperature from −40° C. to 110° C. to produce the ester of formula (59).

(c) The phenyl group of ester (59) is oxidized to a carboxylic acid group by reaction with, sodium periodate and ruthenium (III) chloride in a mixture of carbon tetrachloride or ethyl acetate and acetonitrile to which water is added. The reaction mixture is stirred at a temperature from −40° C. to 80° C. to produce carboxylic acid of formula (60).

(d) The carboxyl group of acid (60) is converted to an ester group by addition

to a mixture of iodomethane in a solvent selected from dichloromethane, chloroform, tetrahydrofuran, toluene or 1,4-dioxane to which a base such as 1,8-diazabicyclo[5.4.0]undec-7-ene (DBU), triethylamine or 1,5-diazabicyclo[4.3.0]non-5-ene (DBN) is added and stirred at a temperature from −40° C. to 110° C. to produce the ester of formula (61); or

to a mixture of methanol and a concentrated acid such as sulphuric acid or hydrochloric acid at a temperature ranging from 0° C. to 100° C.; or

to trimethylsilyldiazomethane and methanol in benzene or toluene at a temperature from −40° C. to 100° C.; or to diazomethane in a solvent such as benzene, toluene, dichloromethane at a temperature from −40° C. to 40° C.

(e) The tert-butoxy group is removed from diester (61) by reaction with trifluoroacetic acid in a solvent e.g. dichloromethane, chloroform, 1,4-dioxane, tetrahydrofuran, diethyl ether, or tert-butyl methyl ether. The reaction mixture is stirred from a temperature from −40° C. to 110° C. to give carboxylic acid of formula (62).

(f) The ester group of acid (62) is converted to isocyanate (63) by addition

to a mixture of a base selected from triethylamine or diisopropylethylamine and a solvent selected from toluene, benzene, xylenes, tetrahydrofuran, diethyl ether or n-heptane to which diphenylphosphoryl azide (DPPA) is added and stirring at a temperature from 0° C. to 150° C.; or

to ethyl chloroformate or isobutyl chloroformate and a base such as triethylamine or diisopropylethylamine in tetrahydrofuran or acetone or diethyl ether at a temperature of −40° C. to 78° C. followed by addition of sodium azide in water and tetrahydrofuran or acetone followed by addition of toluene or benzene and refluxing.

(g) Simultaneous hydrolysis of the isocyanate and ester groups of compound (63) e.g. by aqueous hydrochloric acid at a concentration of from 0.01 M to 12 M in the presence or absence of a solvent such as 1,4-dioxane, acetic acid or water gives the amino acid (64).

Method E:

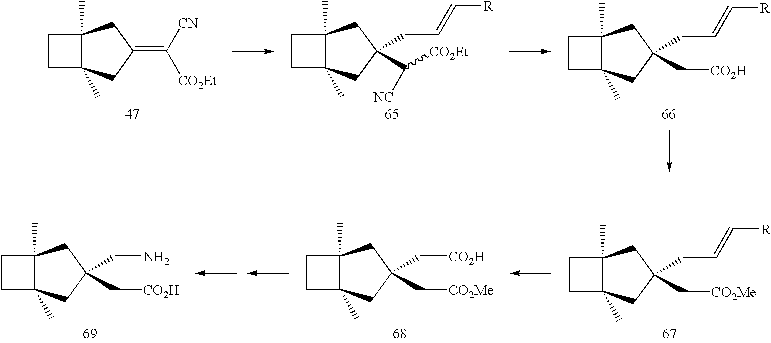

(a) Cyanoester (47) is reacted with allylmagnesium chloride or bromide or 2-butenylmagnesium chloride and a dialkylzinc such as dimethylzinc or a copper (I) salt such as copper (I) iodide or copper (I) cyanide in a dry organic solvent e.g. tetrahydrofuran, 1,4-dioxane, n-heptane, toluene, diethyl ether or tert-butyl methyl ether at a temperature from −100° C. to 110° C. to give an unsaturated addition product of formula (65).

(b) The cyano group of addition product (65) is removed by reaction with a base, e.g. potassium hydroxide, sodium hydroxide, lithium hydroxide or cesium hydroxide in an organic solvent selected from ethylene glycol, 2-methoxyethyl ether, 1,4-dioxane or diethylene glycol. The reaction mixture is stirred at a temperature from 25° C. to 250° C. to give a carboxylic acid of formula (66).

(c) The carboxylic acid group of acid (66) is converted to an ester group by addition

to a mixture of iodomethane in a solvent selected from dichloromethane, chloroform, tetrahydrofuran, toluene or 1,4-dioxane to which a base such as 1,8-diazabicyclo[5.4.0]undec-7-ene (DBU), triethylamine or 1,5-diazabicyclo[4.3.0]non-5-ene (DBN) was added and stirred at a temperature from −40° C. to 110° C. to produce the ester of formula (67); or

to a mixture of methanol and a concentrated acid such as sulphuric acid or hydrochloric acid at a temperature ranging from 0° C. to 100° C.; or

to trimethylsilyldiazomethane and methanol in benzene or toluene at a temperature from −40° C. to 100° C.; or

to diazomethane in a solvent such as benzene, toluene, dichloromethane at a temperature from −40° C. to 40° C.

(d) The unsaturated group in ester (67) is oxidized by sodium periodate and ruthenium (III) chloride in a mixture of carbon tetrachloride or ethyl acetate and acetonitrile to which water is added. The mixture is stirred at a temperature from −40° C. to 80° C. to give a carboxylic acid of formula (68).

(e) Carboxylic acid (68) is converted to amino acid (69) as in method C.

The above ketones can also be transformed into amino acids using one of the following general methods F to G, as illustrated below for ketone of type (9).

Method F

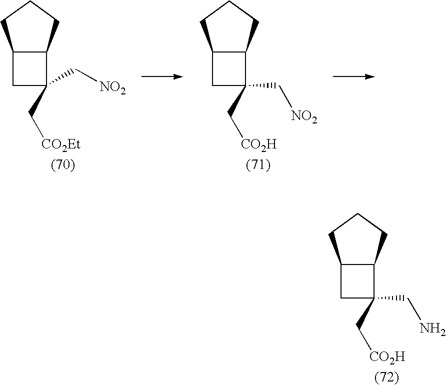

(a) The ketone is converted to the nitro ester (70) according to the methods described hereinabove.

(b) Nitro ester (70) is hydrolysed with a suitable base, such as aqueous sodium hydroxide to give nitro acid (71) which is reduced by suitable hydrogenation, e.g. H₂ on a palladium/carbon catalyst in a suitable solvent, such as ethanol to give the amino acid (72).

Method G

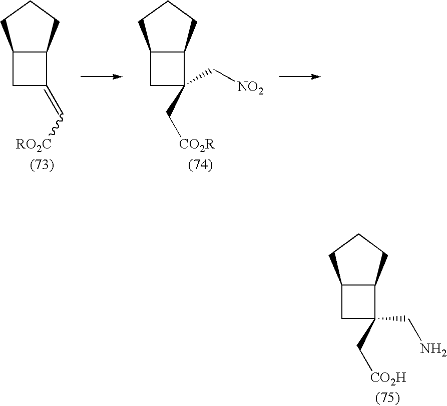

(a) The unsaturated ester (73), where R is benzyl or diphenylmethyl may be prepared from the ketone according to any of the general methods described above.

(b) The nitro ester (74) is converted to the amino acid (75) by reduction by catalytic hydrogenation in a suitable solvent.

Compounds of the invention may alternatively be prepared from the known unsaturated version of a ketone of type (8) as follows in Methods H and I:

Method H

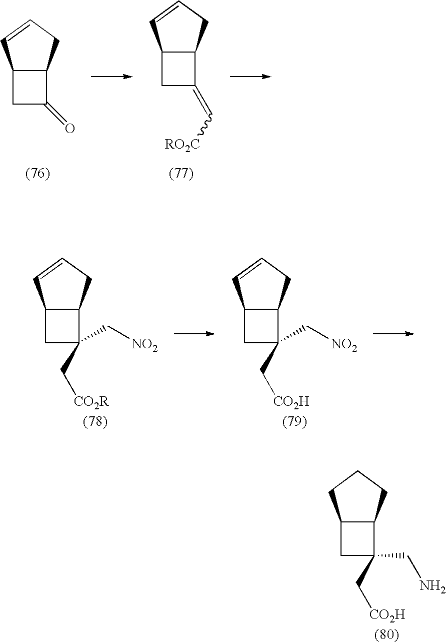

(a) Ketone (76) is converted to the unsaturated nitro ester (78) according to the general methods described hereinabove.

(b) Nitro ester (78) is hydrolysed with a suitable base, such as aqueous sodium hydroxide to give nitro acid (79) which is reduced by hydrogenation, e.g. H₂ on a palladium/carbon catalyst in a suitable solvent, such as ethanol to give the amino acid (80).

Method I

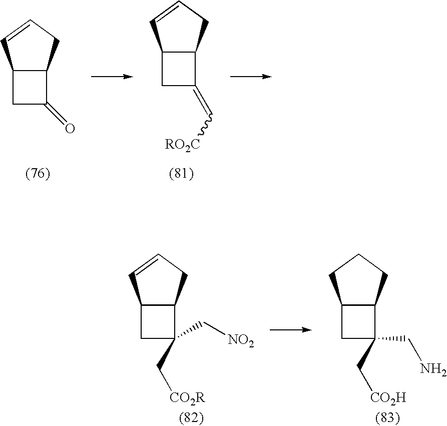

(a) The unsaturated nitro ester (82) may be prepared from the ketone (76) according to the methods generally described hereinabove.

(b) The nitro ester (82) is converted to the amino acid (83) by reduction by catalytic hydrogenation in a suitable solvent.

A pharmaceutically acceptable salt of a compound of the invention may be readily prepared by mixing together solutions of a compound of the invention and the desired acid or base, as appropriate. The salt may precipitate from solution and be collected by filtration or may be recovered by evaporation of the solvent.

Referring to the general methods above, it will be readily understood to the skilled person that where protecting groups are present, these will be generally interchangeable with other protecting groups of a similar nature, e.g. where an acid group is described as being protected with an ethyl group, this may be readily interchanged with any suitable alkyl group, suitably a C₁₋₆alkyl group.

It will be readily understood to the skilled person that particular steps in the general methods presented herein above may be suitably combined in any other manner not shown to provide a compound according to the present invention.

Thus, in summary, the invention provides:-

(i) the use of a compound of the formula I-XXV or of a pharmaceutically acceptable salt, solvate, polymorph, pro-drug or composition thereof, for the manufacture of a medicament for the treatment of fibromyalgia;

(ii) a method of treatment of fibromyalgia in a mammal, including treating said mammal with an effective amount of a compound of the formula I-XXV or with a pharmaceutically acceptable salt, solvate, polymorph, pro-drug or composition thereof; and

(iii) a pharmaceutical composition for the treatment of fibromyalgia comprising a compound of the formula I-XXV or a pharmaceutically acceptable salt, solvate, polymorph or pro-drug thereof and a suitable carrier.

The present invention is illustrated by the following non-limiting examples and intermediates.

### EXAMPLE 1

### [(1R,5R,6S)-6-(Aminomethyl)bicycl[3.2.0]hept-6-yl]acetic acid hydrochloride

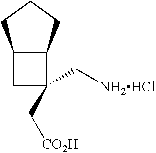

The isocyanate of preparation 9 (approx 9.33 mmol) and 6N hydrochloric acid (30 ml) were refluxed for 18 h. The mixture was allowed to cool, diluted with water (60 ml) and extracted with dichloromethane (2×50 ml). The aqueous phase was concentrated under reduced pressure to give a yellow solid which was washed with ethyl acetate and acetonitrile to give 0.92 g of the title compound as a white solid.

¹H-NMR (400 MHz, d₆-DMSO): δ=7.94 (3H, br s), 3.15 (1H, d), 3.07 (1H, d), 2.72 (1H, quin), 2.46 (1H, m), 2.42 (1H, d), 2.33 (1H, d), 1.98 (1H, m), 1.80-1.64 (2H, m), 1.59 (1H, m), 1.48-1.28 (3H, m), 1.23 (1H, dd). LRMS (APCI): m/z [(MH-HCl)⁺] 184. LCMS (Prodigy ODS3 (3μ) 150 mm×4.6 mmid column, 20-100% Acetonitrile+0.1% formic acid) Retention Time=4.34 min, 100% purity. [α]_{D} (c=0.127 in methanol)=12.4° Microanalysis: Found: C, 54.64; H, 8.19; N, 6.42. C₁₀H₁₇NO₂.HCl requires C, 54.67; H, 8.26; N, 6.38%. Melting Point (Perkin Elmer DSC7): 198° C.

Alternatively:

### EXAMPLE 1A

### [(1R,5R,6S)-6-(Aminomethyl)bicyclo[3.2.0]hept-6-yl]acetic acid hydrochloride

The nitro acid of preparation 32 (2.0 g; 9.4 mmol) in (either 1:1 IPA:H₂O or) 1:1 MeCN:H₂O (40 ml; 20 ml/g) was hydrogenated using 10% Pd/C (0.2 g; 0.1 g/g) at 50° C. and 60 psi for 18 hours. The reaction mixture was filtered through Celite and the filter pad washed with 1:1 IPA:H₂O or 1:1 MeCN:H₂O (20 ml). The combined filtrate and wash were concentrated under vacuum and azeotroped dry with further IPA or MeCN to yield the title compound as a white crystalline solid (1.52 g).

### EXAMPLE 1B

### (1R,5R,6S)-6-(Aminomethyl)bicyclo[3.2.0]hept-6-yl]acetic acid hydrochloride

The lactam of preparation 33 (4.70 g, 28.44 mmol) and hydrochloric acid (57 ml of a 6N solution) were refluxed together for 6 h. The mixture was allowed to cool and then diluted with water (60 ml). The aqueous layer was washed with dichloromethane (2×100 ml), filtered and then evaporated under reduced pressure. The resulting off-white solid was triturated with ethyl acetate and recrystallised using acetonitrile:water 1:1 to give the title compound (4.51 g).

### EXAMPLE 1C

### [(1R,5R,6S)-6-(Aminomethyl)bicyclo[3.2.0]hept-6-yl]acetic acid (Zwitterion)

The amino acid hydrochloride of Example 1 (2.2 g) was dissolved in 7.25 ml H₂O (3.3 ml/g). The solution was adjusted to pH 7.5, initially with about 1.6 ml aq. NaOH, but finally with some drops of aqueous 0.1N aq. NaOH The precipitated zwitterion was stirred for 8 hours at 8° C. and the slurry filtered and the residues washed with ice-cold water (6 ml). The water-wet filter cake was slurried in IPA (15 ml) and refluxed for 10 minutes. After cooling to ambient temperature, the slurry was filtered, and the residues washed with IPA (5 ml). The filter cake was reslurried in IPA (15 ml), refluxed and cooled to ambient temperature. The slurry was filtered and the residues washed with IPA (5 ml) and dried under vacuum at 40° C. to constant weight to yield the title compound as a crystalline solid (1.4 g).

Melting Point (Perkin Elmer DSC7): 208° C.

### EXAMPLE 2

### [(1S,5S,6R)-6-(Aminomethyl)bicyclo[3.2.0]hept-6-yl]acetic acid hydrochloride

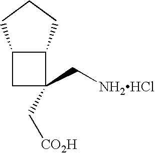

The isocyanate of preparation 12 (approx 11.0 mmol) and 6N hydrochloric acid (30 ml) were refluxed for 16 h. The mixture was allowed to cool, diluted with water (100 ml) and extracted with dichloromethane (2×50 ml). The aqueous phase was concentrated under reduced pressure to give a yellow solid and washed with ethyl acetate and acetonitrile to give 0.94 g of the title compound as a white solid.

¹H-NMR (400 MHz, d₆-DMSO): δ=7.94 (3H, br s), 3.15 (1H, d), 3.07 (1H, d), 2.72 (1H, quin), 2.46 (1H, m), 2.42 (1H, d), 2.33 (1H, d), 1.98 (1H, m), 1.80-1.64 (2H, m), 1.59 (1H, m), 1.48-1.28 (3H, m), 1.23 (1H, dd). LRMS (APCI): m/z [(MH-HCl)⁺] 184. LCMS (Prodigy ODS3 (3μ) 150 mm×4.6 mmid column, 20-100% Acetonitrile +0.1% formic acid) Retention Time=4.34 min, 100% purity. [α]_{D} (c=0.35 in methanol)=+13.0°.

### EXAMPLE 3

### [(1RS,5RS,6RS)-6-(Aminomethyl)bicyclo[3.2.0]hept-6-yl]acetic acid hydrochloride

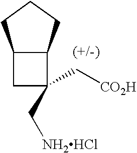

The isocyanate of preparation 17 (approx 2.79 mmol) and 6N hydrochloric acid (15 ml) were refluxed for 18 h. The mixture was allowed to cool, diluted with water (60 ml) and extracted with dichloromethane (3×50 ml). The aqueous phase was concentrated under reduced pressure to give a yellow solid which was washed with ethyl acetate and acetonitrile to give 0.45 g of the title compound as a white solid.

¹H-NMR (400 MHz, d₆-DMSO): δ=7.84 (3H, br s), 2.92 (1H, d), 2.85 (1H, d), 2.75 (1H, t), 2.69 (1H, d), 2.59 (1H, d), 2.39 (1H, t), 1.81-1.62 (4H, m), 1.41-1.30 (4H, m). LRMS (APCI): m/z [(MH-HCl)⁺] 184 LCMS (Prodigy ODS3 (3μ) 150 mm×4.6 mmid column, 20-100% Acetonitrile+0.1 % formic acid) Retention Time=4.27 min, 99.8% purity.

### EXAMPLE 4

### [(1RS,6RS,7SR)-7-(Aminomethyl)bicyclo[4.2.]oct-7-yl]acetic acid hydrochloride

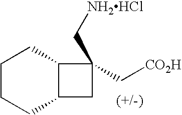

The lactam of preparation 22 (3.20 g, 17.9 mmol) was heated to reflux in 1,4-dioxane (15 ml) and 6N HCl (50 ml). After 4 hrs the mixture was cooled to room temperature and washed with dichloromethane (2×30 ml). The aqueous phase was collected and the solvent removed in vacuo. The residue was triturated with ethyl acetate and the resulting solid collected by filtration and dried under vacuum to give 2.74 g of the title compound as a white solid.

¹H-NMR (400 MHz, D₂O): 3.24 (2H, m), 2.58 (2H, s), 2.39 (1H, m), 2.03 (1H, m), 1.76 (2H, m), 1.59-1.10 (7H, m), 0.96 (1H, m). LRMS (APCI): m/z [(MH-HCl)⁺] 198.

### EXAMPLE 5

### [(1RS,6RS,7RS)-7-(Aminomethyl)bicyclo[4.2.0]oct-7-yl]acetic acid hydrochloride

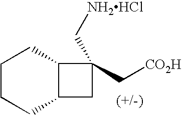

Diphenylphosphoryl azide (0.43 ml, 1.98 mmol) was added to a stirring solution of triethylamine (0.28 ml, 2.03 mmol) and the acid of preparation 29 (0.47 g, 1.96 mmol approx) in toluene (15 ml) at room temperature under nitrogen. The mixture was stirred for 16 hrs and then warmed to 35° C. for 1 hr. The mixture was allowed to cool, diluted with ethyl acetate (60 ml), washed with saturated aqueous sodium hydrogen carbonate (2×100 ml), brine, and dried (MgSO₄). The solvent was removed under reduced pressure and the resulting yellow oil was heated to reflux in 6N HCl (20 ml). After 18 hrs the mixture was cooled to room temperature and washed with dichloromethane (2×60 ml) and diethyl ether (60 ml). The aqueous phase was collected and the solvent removed in vacuo. The residue was triturated with ethyl acetate and the resulting solid collected by filtration and dried under vacuum to give 0.304 g of title compound as a white solid.

¹H-NMR (400 MHz, d₆-DMSO): 3.04 (1H, d), 2.99 (1H, d), 2.68 (1H, d), 2.62 (1H, d), 1.98 (1H, m), 1.83 (1H, t), 1.69-1.28 (9H, m), 1.00 (1H, m). LRMS (APCI): m/z [(MH-HCl)⁺] 198.

### Preparation 1

### (1RS,5RS)-Bicyclo[3.2.0]heptan-6-one

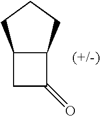

Palladium (1 g, 10% w/w on charcoal) was added to a solution of bicyclo[3.2.0]hept-2-en-6-one (12 ml, 111.3 mmol) in ethyl acetate (100 ml) and the mixture was hydrogenated for 6 hours at 30° C. and 483 kPa (70 p.s.i.). The reaction mixture was filtered and the solvent was evaporated under reduced pressure to give 12.1 g of the title compound as a colourless oil.

vₘₐₓ(film)/cm⁻¹ 1777. ¹H-NMR (400 MHz, CDCl₃): δ=3.54 (1H, m), 3.19 (1H, ddd), 2.88 (1H, m), 2.49 (1H, ddd), 2.04 (1H, m), 1.91-1.49 (5H, m).

### Preparation 1A

### (1R,5R)-bicyclo[3.2.0]heptan-6-one

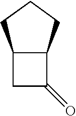

A solution of (1S,5R)-bicyclo[3.2.0]hept-2-en-6-one¹ (50.0 g; 462 mmol) in EtOAc (375 mL) was hydrogenated using 50% wet 5% Pd/C (5.0 g) at 60 psi for 8 hours at ambient temperature. The reaction mixture was filtered through Celite, and the filtrate concentrated under vacuum to yield 41.3 g of the title compound as a colourless oil.

¹H-NMR (400 MHz, CDCl₃): δ=3.55 (1H, m), 3.20 (1H, m), 2.90 (1H, m), 2.50 (1H, m), 2.0-1.5 (6H, m). ¹Ref: EP0074856

### Preparation 2

### Ethyl (2E/Z)-(1RS,5RS)-bicyclo[3.2.0]hept-6-ylidene(cyano)ethanoate

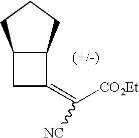

The ketone of preparation 1 (22.4 g, 204.1 mmol), ethyl cyanoacetate (21.7 ml, 204.1 mmol), ammonium acetate (15.7 g, 204.1 mmol) and glacial acetic acid ( 1.7 ml, 204.1 mmol) were refluxed in toluene (220 ml) using a Dean-Stark trap. After 8 h, the mixture was allowed to cool and diluted with ethyl acetate (300 ml), washed with water (3×50 ml), brine and dried (MgSO₄). The solvent was evaporated under reduced pressure. The residue was chromatographed (SiO₂, heptane/ethyl acetate, 95:5 to 7:3) to give 30 g of a 6:4 mixture of isomers of the title compound as a yellow solid.

vₘₐₓ(film)/cm¹ 2225, 1725, 1640. ¹H-NMR (400 MHz, CDCl₃): δ(major isomer)=4.26 (2H, m), 3.64 (1H, m), 3.36 (1H, ddd), 2.96 (1H, m), 2.70 (1H, dt), 2.11 (1H, m), (1.92-1.58, 5H, m), 1.32 (3H, m); δ(minor isomer)=4.26 (2H, m), 3.85 (1H, m), 3.15 (1H, ddd), 2.96 (1H, m), 2.52 (1H, dt, J 20.0, 4.4), 2.02 (1H, m), (1.92-1.58, 5H, m), 1.32 (3H, m). LRMS (APCI): m/z [M-H] 204.

### Preparation 3

### Ethyl [(1RS,5RS,6RS)-6-benzylbicyclo[3.2.0]hept-6-yl](cyano)acetate

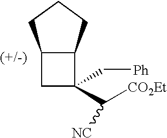

The cyanoester of preparation 2 (10.0 g, 48.7 mmol) in THF (60 ml) was added over 1 h to a stirring solution of benzylmagnesium chloride (78 ml of a 1M solution in ether, 78 mmol) in THF (100 ml) at −78° C. under argon. After stirring for 2 h at this temperature, the mixture was quenched by addition of saturated ammonium chloride solution (40 ml). The mixture was allowed to warm to room temperature, and dilute hydrochloric acid (150 ml) was added. The aqueous layer was extracted with ethyl acetate (3×100 ml). The combined organic layers were washed with brine, dried (MgSO₄) and the solvent was evaporated under reduced pressure to give the title compound as a mixture of diastereoisomers and as a yellow oil which was used crude in the next step.

vₘₐₓ(film)/cm⁻¹ 2247, 1741. LRMS (APCI): m/z [M-H] 296.

### Preparation 4

### [(1RS,5RS,6SR)-6-benzylbicyclo[3.2.0]hept-6-yl]acetic acid

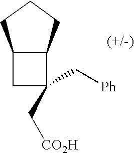

The mixture of the diastereomeric cyano-esters of preparation 3 (20.3 g, 68.4 mmol) and potassium hydroxide (23.0 g, 410.4 mmol) were heated to 160° C. in ethylene glycol (350 ml) for 38 h. After this time, the mixture was allowed to cool and dilute hydrochloric acid (300 ml) was added carefully. The mixture was extracted with ethyl acetate (3×200 ml) and the combined organic fractions were washed with brine, dried (MgSO₄) and the solvent was evaporated under reduced pressure. The residue was chromatographed (SiO₂, heptane/ethyl acetate, 8:2) to give 14.6 g of the racemic diastereomeric title compound as a white solid.

vₘₐₓ(film)/cm⁻¹ 3344, 1704. ¹H-NMR (400 MHz, CDCl₃): δ=7.31-7.22 (5H, m), 3.02 (1H, d), 2.97 (1H, d), 2.64 (2H, m), 2.34 (1H, d), 2.24 (1H, d), 2.13 (1H, m), 1.84-1.59 (3H, m), 1.50-1.32 (4H, m). LRMS (APCI): m/z [M-H] 243.

### Preparation 5

### [(1R,5R,6S)-6-benzylbicyclo[3.2.0]hept-6-yl]acetic acid

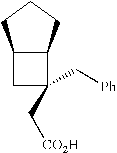

(R)-(+)-α-Methylbenzylamine (6.67 g, 55 mmol) was added to a stirring solution of racemic acid of preparation 4 (24 g, 98.2 mmol) dissolved in ethyl acetate. The acid salt precipitated out of the solution as a white solid. This was recrystallised three times from ethyl acetate to give 8.5 g of the acid salt. Further recrystallisation of the residue gave an additional batch of 8.5 g of the acid salt. The first batch of the salt was taken up in dichloromethane, washed with dilute hydrochloric acid, brine and dried (MgSO₄). The solvent was evaporated under reduced pressure to give 5.0 g of the title compound as a white solid.

HPLC [Chiralcel OD 250×4.6 mm column (Mobile phase: 90% hexane, 10% IPA cont. 0.5% TFA)]: Retention time=5.1 min (94% ee). [α]_{D} (c=1.13 in chloroform)=−20.2°.

The second batch of the salt was taken up in dichloromethane, washed with dilute hydrochloric acid, brine and dried (MgSO₄) to give a further 5 g of acid of 86% ee.

Similarly prepared was:

### Preparation 6

### [(1S,5S,6R)-6-benzylbicyclo[3.2.0]hept-6-yl]acetic acid

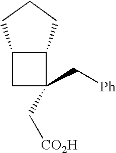

by recrystallisation of the salt generated by addition of (S)-(−)-α-methylbenzylamine. HPLC [Chiralcel OD 250×4.6 mm column (Mobile phase: 90% hexane, 10% IPA cont. 0.5% TFA)]: Retention time=4.2 min (95% ee). [α]_{D} (C=1.0 in chloroform)=+17.3°.

### Preparation 7

### Methyl [(1R,5R,6S)-6-benzylbicyclo[3.2.0]hept-6-yl]acetate

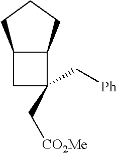

Trimethylsilyldiazomethane (17.7 ml of a 2M solution in hexane, 35.4 mmol) was added dropwise to a stirring solution of acid of preparation 5 (7.85 g, 32.1 mmol) in a mixture of toluene (90 ml) and methanol (22.5 ml) at 0° C. under argon. The mixture was allowed to warm to room temperature and stirred for 4 h. The solvent was removed under reduced pressure and the residue was taken up in ethyl acetate (150 ml), washed with saturated sodium hydrogen carbonate (150 ml), dilute hydrochloric acid (100 ml), brine and dried (MgSO₄). The solvent was evaporated under reduced pressure The residue was chromatographed (SiO₂, heptane/ethyl acetate, 9:1) to give 7.0 g of the title compound as a colourless oil.

vₘₐₓ(film)/cm⁻¹ 1736. ¹H-NMR (400 MHz, CDCl₃): δ=7.28-7.21 (5H, m), 3.67 (3H, s), 2.97 (1H, d), 2.92 (1H, d,), 2.65-2.60 (2H, m), 2.26 (1H, d), 2.18 (1H, d), 2.08 (1H, m), 1.82-1.52 (3H, m), 1.48-1.22 (4H, m). LRMS (APCI): m/z [MH⁺] 259. [α]_{D} (c=0.11 in methanol)=-24.1°.

### Preparation 8

### [(1R,5R,6S)-6-(2-methoxy-2-oxoethyl )bicyclo[3.2.0]hept-6-yl]acetic acid

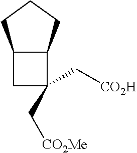

The ester of preparation 7 (7.0 g, 27.1 mmol) and sodium periodate (81.1 g, 379.3 mmol) were stirred together in ethyl acetate (100 ml), acetonitrile (100 ml) and water (150 ml) for 5 minutes. The mixture was cooled to 0° C. and ruthenium (III) chloride hydrate (0.11 g, 0.54 mmol) was added to the reaction mixture. The reaction was allowed to warm to room temperature and stirred for 24 h. Diethyl ether (150 ml) was added and the mixture was stirred for 40 minutes. Dilute hydrochloric acid (200 ml) was added to the mixture which was then extracted with ethyl acetate (3×100 ml). The combined organic fractions were washed with saturated sodium thiosulfate solution, brine, dried (MgSO₄) and the solvent was evaporated under reduced pressure to give the title compound as a yellow oil.

vₘₐₓ(film)/cm⁻¹ 1733, 1715. ¹H-NMR (400 MHz, CDCl₃): δ=3.65 (3H, s), 2.82-2.76 (3H, m), 2.55-2.49 (3H, m), 2.05 (1H, m), 1.81 (1H, m), 1.73-1.69 (2H, m), 1.49-1.28 (4H, m). LRMS (APCI): m/z [M-H] 225.

### Preparation 9

### Methyl [(1R,5R,6S)-6-(Isocyanatomethyl)bicyclo[3.2.0]hept-6-yl]acetate

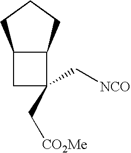

Diphenylphosphoryl azide (8.45 ml, 39.2 mmol) was added to a stirring solution of triethylamine (5.6 ml, 40.4 mmol) and the acid of preparation 8 (8.78 g, 38.8 mmol) in toluene (80 ml) at room temperature under nitrogen. The mixture was stirred for 3 hours and then warmed to 35° C. for 1.5 hours. The mixture was allowed to cool, diluted with ethyl acetate (150 ml), washed with saturated aqueous sodium hydrogen carbonate (150 ml), brine, and dried (MgSO₄). The solvent was removed under reduced pressure to give 8.7 g of the title compound as a yellow oil.

vₘₐₓ (film)/cm⁻¹ 2265, 2171, 1733.

### Preparation 10

### Methyl [(1S,5S,6R)-6-benzylbicyclo[3.2.01]hept-6-yl]acetate

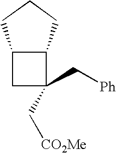

Trimethylsilyldiazomethane (5.7 ml of a 2M solution in hexane, 11.4 mmol) was added dropwise to a stirring solution of the acid of preparation 6 (2.77 g, 11.3 mmol) in a mixture of toluene (30 ml) and methanol (7.5 ml) at 0° C. under argon. The mixture was allowed to warm to room temperature and stirred for 4 h. The solvent was removed under reduced pressure and the residue was taken up in ethyl acetate (100 ml), washed with saturated sodium hydrogen carbonate (100 ml), dilute hydrochloric acid (100 ml), brine and dried (MgSO₄). The solvent was evaporated under reduced pressure The residue was chromatographed (SiO₂, heptane/ethyl acetate, 9:1) to give 2.84 g of the title compound as a colourless oil.

¹H-NMR (400 MHz, CDCl₃): δ=7.28-7.21 (5H, m), 3.67 (3H, s), 2.97 (1H, d), 2.92 (1H, d,), 2.65-2.60 (2H, m), 2.26 (1H, d), 2.18 (1H, d), 2.08 (1H, m), 1.82-1.52 (3H, m), 1.48-1.22 (4H, m); [α]_{D} (C=0.11 in methanol)=+23.1°.

### Preparation 11

### [(1S,5S,6R)-6-(2-methoxy-2-oxoethyl)bicyclo[3.2.0]hept-6-yl]acetic acid

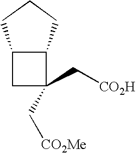

The ester of preparation 10 (7.0 g, 27.1 mmol) and sodium periodate (81.1 g, 379.3 mmol) were stirred together in ethyl acetate (100 ml), acetonitrile (100 ml) and water (150 ml) for 5 minutes. The mixture was cooled to 0° C. and ruthenium (III) chloride hydrate (0.11 g, 0.54 mmol) was added to the reaction mixture. The reaction was allowed to warm to room temperature and stirred for 24 h. Diethyl ether (150 ml) was added and the mixture was stirred for 40 minutes. Dilute hydrochloric acid (200 ml) was added to the mixture which was then extracted with ethyl acetate (3×100 ml). The combined organic fractions were washed with saturated sodium thiosulfate solution, brine, dried (MgSO₄) and the solvent was evaporated under reduced pressure to give the title compound as a yellow oil.

¹H-NMR (400 MHz; CDCl₃): δ=3.65 (3H, s), 2.82-2.76 (3H, m), 2.55-2.49 (3H, m), 2.05 (1H, m), 1.81 (1H, m), 1.73-1.69 (2H, m), 1.49-1.28 (4H, m).

### Preparation 12

### Methyl [(1S,5S,6R)-6-(isocyanatomethyl)bicyclo[3.2.0]hept-6-yl]acetate

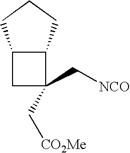

Diphenylphosphoryl azide (2.4 ml, 11.1 mmol) was added to a stirring solution of triethylamine (1.6 ml, 11.4 mmol) and the acid of preparation 11 (11.0 mmol approx) in toluene (30 ml) at room temperature under nitrogen. The mixture was refluxed for 2 hours. The mixture was allowed to cool, diluted with ethyl acetate (150 ml), washed with saturated aqueous sodium hydrogen carbonate (2×150 ml), brine, and dried (MgSO₄). The solvent was removed under reduced pressure to give the title compound as a yellow oil.

vₘₐₓ(film)/cm⁻¹ 2265, 2151, 1734.

### Preparation 13

### tert-butyl [(1RS,5RS,6SR)-6-benzylbicyclo[3.2.0]hept-6-yl]acetate

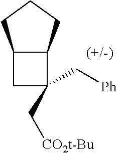

Oxalyl chloride (0.92 ml, 10.5 mmol) was added dropwise to a stirring solution of the acid of preparation 4 (2.34 g, 9.58 mmol) in dichloromethane (30 ml) under argon at 0° C. Dimethylformamide (0.3 ml) was carefully added and the mixture was allowed to warm to room temperature and stirred for a further 4 hours. The solvent was removed in vacuo and the residue diluted with dichloromethane (20 ml). 2-Methyl propan-1-ol (10 ml) in dichloromethane (20 ml) was carefully added to the reaction mixture under argon followed by diisopropylethylamine (2.5 ml, 14.4 mmol). The mixture was stirred for 17 hours and then taken up in ethyl acetate, washed with saturated aqueous sodium hydrogen carbonate (2×200 m¹), and dried (MgSO₄). The solvent was removed under reduced pressure and the residue was chromatographed (SiO₂, heptane/ethyl acetate 95:5) to give the title compound (2.40 g) as a yellow oil. vₘₐₓ(film)/cm⁻¹ 1727. ¹H-NMR (400 MHz, CDCl₃): δ=7.28-7.21 (5H, m, Ph), 2.98 (1H, d), 2.92 (1H, d), 2.64-2.56 (2H, m), 2.16 (1H, d), 2.09 (1H, d), 2.04 (1H, m), 1.80-1.50 (3 H, m), 1.48 (9H, s), 1.47-1.20 (4H, m).

### Preparation 14

### [(1RS,5RS,6SR)-6-(2-tert-Butoxy-2-oxoethyl)bicyclo[3.2.0]hept-6-yl]acetic acid

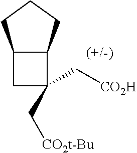

The ester of preparation 13 (2.4 g, 7.99 mmol) and sodium periodate (23.93 g, 111.8 mmol) were stirred together in ethyl acetate (24 ml), acetonitrile (24 ml) and water (36 ml) for 5 minutes. The mixture was cooled to 0° C. and ruthenium (III) chloride hydrate (0.033 g, 0.16 mmol) was added to the reaction mixture. The reaction was allowed to warm to room temperature and stirred for 24 h. Diethyl ether (60 ml) was added and the mixture was stirred for 40 minutes. Dilute hydrochloric acid (150 ml) was added to the mixture which was then extracted with ethyl acetate (3×100 ml). The combined organic fractions were washed with brine, dried (MgSO₄) and the solvent was evaporated under reduced pressure to give the title compound (1.78 g, 83%) as a yellow oil. vₘₐₓ(film)/cm⁻¹ 1728, 1714. ¹H-NMR (400 MHz, CDCl₃): δ=2.78 (1H, d), 2.71 (1H, d), 2.43 (1H, d), 2.38 (1H, d), 2.01 (1H, m), 1.86-1.64 (3H, m), 1.52-1.36 (6H, m), 1.45 (9H, s). LRMS (APCI): m/z [M-H] 267.

### Preparation 15

### [(1RS,5RS,6SR)-6-(2-tert-Butoxy-2-oxoethyl)bicyclo[3.2.0]hept-6-yl]acetic acid methyl ester

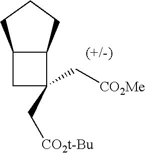

Trimethylsilyldiazomethane (4.3 ml of a 2M solution in hexane, 8.6 mmol) was added dropwise to a stirring solution of the acid of preparation 14 (1.78 g, 6.63 mmol) in a mixture of toluene (24 ml) and methanol (6 ml) at 0° C. under argon. The mixture was allowed to warm to room temperature and stirred for 24 h. The solvent was removed under reduced pressure and the residue was taken up in ethyl acetate (100 ml), washed with saturated sodium hydrogen carbonate (100 ml), dilute hydrochloric acid (100 ml), brine and dried (MgSO₄). The solvent was evaporated under reduced pressure to give the title compound as a yellow oil.

vₘₐₓ(film)/cm⁻¹ 1732. LRMS (APCI): m/z [M-O^{t}Bu] 209.

### Preparation 16

### [(1RS,5RS,6RS)-6-(2-Methoxy-2-oxoethyl)bicyclo[3.2.0]hept-6-yl]acetic acid

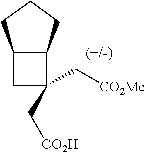

Trifluoroacetic acid (5 ml) was added dropwise to a stirring solution of the ester of preparation 15 (approx. 6.63 mmol) in dichloromethane (15 ml) at 0° C. The mixture was allowed to warm to room temperature and stirred for a further 17 hours. The mixture was washed with saturated aqueous sodium hydrogen carbonate solution until it reached neutral pH and extracted with dichloromethane (50 ml). It was then reacidified to pH 4 with dilute hydrochloric acid. The mixture was then further extracted with dichloromethane (2×50 ml). The combined organic fractions were washed with brine, dried (MgSO₄) and the solvent removed under reduced pressure. The residue was purified by chromatography (SiO₂, 8:2 to 6:4 heptane/ethyl acetate) to give 0.63 g of the title compound as a colourless oil.

vₘₐₓ(film)/cm⁻¹ 3200, 1738, 1705. ¹H-NMR (400 MHz, CDCl₃): δ=3.68 (3H, s), 2.84-2.73 (3H, m), 2.61-2.48 (3H, m), 2.03 (1H, m), 1.80 (1H, m), 1.79-1.32 (6H, m). LRMS (APCI): m/z [M-H] 225.

### Preparation 17

### Methyl [(1RS,5RS,6RS)-6-(isocyanatomethyl)bicyclo[3.2.0]hept-6-yl]acetate

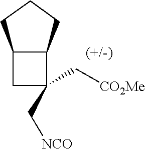

Diphenylphosphoryl azide (0.61 ml, 2.82 mmol), triethylamine (0.40 ml, 2.90 mmol), and the acid of preparation 16 (0.63 g, 2.79 mmol) were refluxed in toluene (15 ml) for 6 h. The mixture was allowed to cool and diluted with ethyl acetate (60 ml). The resulting solution was washed with saturated aqueous sodium hydrogen carbonate (150 ml), brine, and dried (MgSO₄). The solvent was removed under reduced pressure to give the title compound as a yellow oil.

R_{f}heptane-ethyl acetate, 9:1) 0.36. vₘₐₓ (film)/cm⁻¹ 2259, 2171, 1736.

### Preparation 18

### (1RS,6SR)-8,8-Dichlorobicyclo[4.2.0]octan-7-one

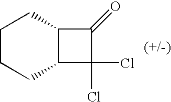

Copper (II) sulphate (2.0 g, 8.0 mmol) was dissolved in water (75 ml) and added to zinc dust (30 g). The mixture was stirred for 2 hours. The mixture was filtered and the solid collected, washed twice with acetone and dried under vacuum at 100° C. for 24 hrs. A portion of the activated zinc (8.0 g) was added to a solution of cyclohexene (10 ml, 98.9 mmol) in diethyl ether (180 ml). Trichloroacetyl chloride (10.48 ml, 93.96 mmol) in diethyl ether (20 ml) was added at such a rate to keep the mixture at reflux. After the addition was complete, the mixture was heated to reflux for 4 hrs. The mixture was cooled to room temperature, diluted with diethyl ether (50 ml) and carefully poured into an aqueous saturated solution of sodium bicarbonate. The mixture was acidified with 2N HCl and the organic phase separated. The ether extract was washed with water and then with saturated aqueous sodium bicarbonate. The organic phase was collected, dried (MgSO₄) and the solvent removed under reduced pressure. The residue was purified by flash chromatography (silica, EtOAc:Heptane 1:9) to give 8.62 g of the title compound as a clear oil.

vₘₐₓ (film)/cm⁻¹ 2939, 1802. ¹H-NMR (400 MHz, CDCl₃): δ=3.94 (1H, m), 2.95 (1H, m), 2.18-1.82 (2H, m), 1.80-1.20 (6H, m).

### Preparation 19

### (1RS,6RS)-Bicyclo[4.2.0]octan-7-one

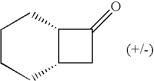

(1RS,6SR)-8,8-dichlorobicyclo[4.2.0]octan-7-one (preparation 18) (8.60 g, 44.6 mmol) was heated to reflux in acetic acid (100 ml) with zinc dust (29.0 g, 446 mmol). After 4 hrs the mixture was cooled to room temperature, diluted with diethyl ether (200 ml) and washed with 2N NaOH (2×100 ml) and then with saturated aqueous NaHCO₃ (4×100 ml). The ether phase was collected, dried (MgSO₄) and the solvent was removed under reduced pressure to give 4.79 g of the title compound as a clear oil.

vₘₐₓ(film)/cm⁻¹ 2930, 1776. ¹H-NMR (400 MHz, CDCl₃): δ=3.27 (1H, m), 3.12 (1H, m), 2.42 (2H, m), 2.20-1.02 (8H, m).

### Preparation 20

### Ethyl (2Z/E)-(1RS,6RS)-bicyclo[4.2.0]oct-7-ylideneethanoate

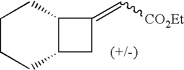

Sodium hydride (60% dispersion in oil, 1.46 g, 36.6 mmol) was suspended in dry tetrahydrofuran (150 ml) and cooled to 0° C. Triethylphosphonoacetate (7.65 ml, 38.5 mmol) was added and the mixture stirred at 0° C. for 15 mins. A solution of (1RS,6RS)-bicyclo[4.2.0]octan-7-one (preparation 19) (4.78 g, 38.5 mmol) in THF (20 ml) was then added and the mixture stirred at 0° C. After 1 hr the mixture was allowed to warm to room temperature, diluted with ethyl acetate (200 ml) and washed with 2N HCl (2×150 ml). The organic phase was collected, dried (MgSO₄) and the solvent removed under reduced pressure. The residue was purified by flash chromatography (Silica, EtOAc:Heptane 3:20) to give 5.49 g of the title compound as a clear oil.

vₘₐₓ(film)/cm⁻¹ 2929, 1715, 1186. ¹H-NMR (400 MHz, CDCl₃): δ=5.63 and 5.58 (1H in total—E/Z isomers, 2 x m), 4.15 (2H, m), 3.38-2.98 (2H, m), 2.79-2.35 (2H, m), 2.13-1.05 (1H, m) LRMS (APCI): m/z [MH⁺] 195.

### Preparation 21

### Ethyl [(1RS,6RS,7SR)-7-(nitromethyl)bicyclo[4.2.0]oct-7-yl]acetate

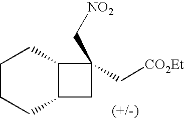

(2Z/E)-(1RS,6RS)-Bicyclo[4.2.0]oct-7-ylideneethanoate (preparation 20) (5.47 g, 28.2 mmol) was heated to 60° C. in tetrahydrofuran (50 ml) with nitromethane (3.05 ml, 56.4 mmol) and tetrabutylammonium fluoride (1M in THF, 42 ml, 42.0 mmol). After 18 hrs the mixture was cooled to room temperature, diluted with ethyl acetate (200 ml) and washed with 2N HCl (2×100 ml) and then with brine. The organic phase was collected, dried (MgSO₄) and the solvent removed in vacuo. The residue was purified by flash chromatography (silica, EtOAc:heptane 1:9) to give 4.73 g of the title compound as a clear oil.

vₘₐₓ(film)/cm⁻¹ 1182, 1547, 1731, 2936. ¹H-NMR (400 MHz, CDCl₃): δ=4.83 (2H, m), 4.12 (2H, q), 2.66 (2H, m), 2.57 (1H, m), 2.22 (1H, m), 2.05 (1H, m), 1.86 (1H, m), 1.76-1.31 (7H, m), 1.26 (3H, t), 1.10(1H, m). LRMS (APCI): m/z [MH⁺] 256.

### Preparation 22

### (1S,6S,7R)-Spiro[bicyclo[4.2.0]octane-7,3′-pyrrolidin]-5′-one

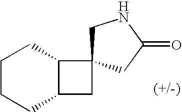

Ethyl [(1RS,6RS,7SR)-7-(nitromethyl)bicyclo[4.2.0]oct-7-yl]acetate (preparation 21) (4.70 g, 18.4 mmol) was shaken in methanol (150 ml) at 30° C. over Raney Nickel catalyst under an atmosphere of hydrogen gas at 483 kPa (70 p.s.i.). After 4 hrs the catalyst was removed by filtration through celite and the solvent removed under reduced pressure to give 3.23 g of the title compound as a clear oil which solidified on standing.

vₘₐₓ(film)/cm⁻¹ 2919, 1712, 1677. ¹H-NMR (400 MHz, CDCl₃): δ=5.61 (1H, br. s), 3.46 (2H, m), 2.42 (2H, m), 2.18-1.01 (12H, m). LRMS (APCI): m/z [MH⁺] 180.

### Preparation 23

### Ethyl (2E/Z)-(1RS,6RS)-bicyclo[4.2.0]oct-7-ylidene(cyano)ethanoate

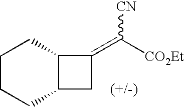

The ketone of preparation 19 (2.85 g, 23.0 mmol), ethyl cyanoacetate (2.45 ml, 23.0 mmol), ammonium acetate (1.77 g, 23.0 mmol) and glacial acetic acid (1.32 ml) were refluxed in toluene (40 ml) using a Dean-Stark trap. After 6 h, the mixture was allowed to cool and diluted with ethyl acetate (150 ml), washed with water (50 ml), brine and dried (MgSO₄). The solvent was evaporated under reduced pressure. The residue was chromatographed (SiO₂, heptane/ethyl acetate, 4:1) to give 2.76 g of a mixture of cyano-esters as a yellow solid.

¹H-NMR (400 MHz, CDCl₃): δ(major isomer); 4.26 (2H, q), 3.36 (1H, m), 3.02 (2H, m), 2.58 (1H, m), 1.30-2.18 (8H, m), 1.33 (3H, t). δ(minor isomer)=4.25 (2H, q), 3.48 (1H, m), 3.23 (2H, m), 2.58 (1H, m), 1.30-2.18 (8H, m), 1.32 (3H, t).

### Preparation 24

### Ethyl [(1RS,6RS,7RS)-7-benzylbicyclo[4.2.0]oct-7-yl](cyano)acetate

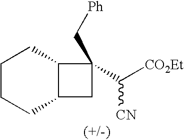

The cyanoester of preparation 23 (2.75 g, 12.5 mmol) in THF (60 ml) was added over 1 h to a stirring solution of benzylmagnesium chloride (20 ml of a 1M solution in ether, 20 mmol) in THF (20 ml) at −78° C. under argon. After stirring for 2 h at this temperature, the mixture was quenched by addition of saturated ammonium chloride solution (10 ml). The mixture was allowed to warm to room temperature, and dilute hydrochloric acid (30 ml) was added. The aqueous layer was extracted with ethyl acetate (3×40 ml). The combined organic layers were washed with brine, dried (MgSO₄) and the solvent was evaporated under reduced pressure to give a mixture of diastereomeric cyano-esters. The residue was chromatographed (SiO₂, heptane/ethyl acetate, 4:1) to give 3.53 g of a mixture of diastereomeric cyano-esters as a clear oil.

R_{f}heptane-ethyl acetate, 4:1)=0.30 vₘₐₓ(film)/cm⁻¹ 2247, 1740.

### Preparation 25

### [(1RS,6RS,7SR)-7-benzylbicyclo[4.2.0]oct-7-yl]acetic acid

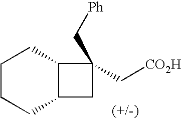

The mixture of diastereomeric cyano-esters of preparation 24 (3.52 g, 11.3 mmol) and potassium hydroxide (3.8 g, 67.9 mmol) were heated to 160° C. in ethylene glycol (75 ml) for 72 h. After this time, the mixture was allowed to cool and dilute hydrochloric acid was added carefully until the solution was acidic by pH paper. The mixture was extracted with ethyl acetate (3×100 ml) and the combined organic fractions were washed with brine, dried (MgSO₄) and the solvent was evaporated under reduced pressure. The residue was chromatographed (SiO₂, ethyl acetate:heptane 1:4) to give 2.11 g of the racemic diastereomeric acid as a yellow oil.

¹H-NMR (400 MHz, CDCl₃): δ=7.31-7.22 (5H, m), 3.08 (1H, d), 3.00 (1H, d), 2.56 (1H, m), 2.44 (1H, d), 2.38 (1H, d), 2.25 (1H, m), 1.98 (1H, m), 1.75 (1H, t), 1.71-1.30 (7H, m), 1.10 (1H, m). LRMS (ES-): m/z [M-H] 257.

### Preparation 26

### tert-butyl [(1RS,6RS 7SR)-7-benzylbicyclo[4.2.0]oct-7-yl]acetate

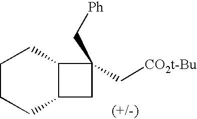

Oxalyl chloride (0.67 ml, 7.62 mmol) was added dropwise to a stirring solution of the acid of preparation 25 (1.79 g, 6.93 mmol) in dichloromethane (25 ml) under nitrogen at 0° C. Dimethylformamide (0.25 ml) was carefully added and the mixture was allowed to warm to room temperature and stirred for a further 4 hours. The solvent was removed in vacuo and the residue diluted with dichloromethane (20 ml). 2-Methyl propan-1-ol (9 ml) in dichloromethane (20 ml) was carefully added to the reaction mixture under argon followed by diisopropylethylamine (1.8 ml, 10.4 mmol). The mixture was stirred for 18 hours and then saturated aqueous sodium hydrogen carbonate (30 ml) was added. The mixture was extracted with ethyl acetate (3×50 ml) and the combined organic fractions were washed with brine and dried (MgSO₄). The solvent was removed under reduced pressure and the residue was chromatographed (SiO₂, heptane/ethyl acetate 98:2) to give ester (2.42 g).

¹H-NMR (400 MHz, CDCl₃): δ=7.33-7.19 (5H, m), 3.05 (1H, d), 2.96 (1H, d), 2.53 (1H, m), 2.30-2.18 (3H, m), 1.90 (1H, m), 1.72 (1H, t), 1.65-1.55 (2H, m), 1.48 (9H, s), 1.47-1.00 (6H, m).

### Preparation 27

### [(1RS,6RS,7SR)-7-(2-tert-Butoxy-2-oxoethyl)bicyclo[4.2.0]oct-7-yl]acetic acid

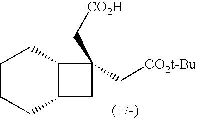

The ester of preparation 26 (6.93 mmol) and sodium periodate (20.75 g, 97.02 mmol) were stirred together in ethyl acetate (20 ml), acetonitrile (20 ml) and water (30 ml) for 5 minutes. The mixture was cooled to 0° C. and ruthenium (III) chloride hydrate (0.03 g, 0.14 mmol) was added to the reaction mixture. The reaction was allowed to warm to room temperature and stirred for 24 h. Diethyl ether (100 ml) was added and the mixture was stirred for 40 minutes. Dilute hydrochloric acid (150 ml) was added to the mixture which was then extracted with ethyl acetate (3×100 ml). The combined organic fractions were washed with brine, dried (MgSO₄) and the solvent was evaporated under reduced pressure to give 0.64 g of acid.

¹H-NMR (400 MHz, CDCl₃): δ=2.84 (1H, d), 2.75 (1H, d), 2.61-2.48 (3H, m), 2.17 (1H, m), 1.95-1.80 (3H, m), 1.78-1.30 (7H, m), 1.44 (9H, s).

### Preparation 28

### [(1RS,6RS,7SR)-6-(2-tert-Butoxy-2-oxoethyl)bicyclo[4.2.0]oct-7-yl]acetic acid methyl ester

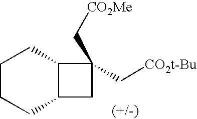

Trimethylsilyldiazomethane (1.2 ml of a 2M solution in hexane, 2.4 mmol) was added dropwise to a stirring solution of the acid of preparation 27 (0.64 g, 2.28 mmol) in a mixture of toluene (10 ml) and methanol (2.5 ml) at 0° C. under argon. The mixture was allowed to warm to room temperature and stirred for 16 h. The solvent was removed under reduced pressure and the residue was taken up in ethyl acetate (150 ml), washed with saturated sodium hydrogen carbonate (100 ml), dilute hydrochloric acid (100 ml), brine and dried (MgSO₄). The solvent was evaporated under reduced pressure to give 0.65 g of ester as a yellow oil.

¹H-NMR (400 MHz, CDCl₃): δ=3.66 (3H, s), 2.83 (1H, d), 2.74 (1H, d), 2.57 (1H, d), 2.49 (1H, d), 2.15 (1H, m), 1.94-1.78 (3H, m), 1.72-1.06 (8H, m), 1.43 (9H, s).

### Preparation 29

### [(1RS,6RS,7SR)-7-(2-Methoxy-2-oxoethyl)bicyclo[4.2.0]oct-7-yl]acetic acid

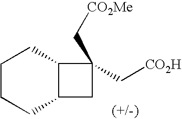

Trifluoroacetic acid (3 ml) was added dropwise to a stirring solution of the ester of preparation 28 (0.65 g, 2.19 mmol) in dichloromethane (9 ml) at 0° C. The mixture was allowed to warm to room temperature and stirred for a further 16 hours. The mixture was washed with saturated aqueous sodium hydrogen carbonate solution and then extracted with ethyl acetate (50 ml). The aqueous layer was acidified to pH 4 with dilute hydrochloric acid and then extracted with ethyl acetate (2×50 ml). The combined organic fractions were washed with brine, dried (MgSO₄) and the solvent removed under reduced pressure. The residue was purified by chromatography (SiO₂, 6:4 heptane/ethyl acetate) to give 0.47 g of acid as a yellow oil.

¹H-NMR (400 MHz, CDCl₃): δ=3.67 (3H, s), 2.84 (1H, d), 2.78 (1H, d), 2.74 (1H, d), 2.66 (1H, d), 2.49 (1H, m), 2.14 (1H, m), 1.95-1.81 (2H, m), 1.70 (1H, m), 1.63 (1H, m), 1.55-1.30 (5H, m), 1.07 (1H, m).

### Preparation 30

### Ethyl (2E)-(1R,5R)-bicyclo[3.2.0]hept-6-ylidene acetate/ethyl (2Z)-(1R,5R)bicyclo[3.2.0]hept-6-ylidene acetate

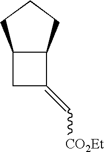

A solution of triethylphosphonoacetate (53.4 g; 238.3 mmol) in THF (25 mL) was added to a suspension of 60% sodium hydride dispersion (9.53 g; 238.3 mmol) in THF (75 mL) maintaining the temperature between 5-15° C. A solution of (1R,5R)-bicyclo[3.2.0]heptan-6-one (preparation 1A) (25 g, 226.9 mmol) in THF (150 ml) was added maintaining the temperature between 5-15° C. The reaction mixture was stirred at ambient temperature for 30 minutes then water (100 mL) added. The phases were separated and the organic layer containing the title compound was used directly in the next step.

¹H-NMR (400 MHz, CDCl₃): δ=5.55 (1H, d), 4.15 (2H, q), 3.40 (1H, m), 3.20 (1H, m), 2.90 (1H, m), 2.55 (1H, m), 1.8-1.5 (5H, m), 1.30 (3H, t).

### Preparation 31

### Ethyl (1R,5R,6S)-[6-(nitromethyl)bicyclo[3.2.0]hept-6-yl]acetate

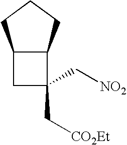

The THF solution of the compound of preparation 30 (assuming 40.9 g of compound in a total volume of 225 mL) was diluted with THF (270 ml). TBAF.3H₂O (93.1 g; 295.0 mmol) and MeNO₂ (453.9 mmol) were added and the solution heated at reflux for 4 hours. The reaction mixture was cooled and concentrated under reduced pressure. Toluene (330 mL) was added and the biphasic mixture washed with water (165 mL), 2M aq. HCl (165 mL+100 mL) and then further water (165 mL). The product-containing toluene layer was dried over MgSO₄ and concentrated under reduced pressure to give the title compound as a red/brown oil (90% (over 2 steps)).

¹H-NMR (400 MHz, CDCl₃): δ=4.80 (2H, m), 4.15 (2H, m), 2.85 (1H, m), 2.65 (1H, m), 2.55 (2H, m), 2.20 (1H, m), 1.9-1.4 (7H,m), 1.25 (3H, t).

### Preparation 32

### (1R,5R,6S)-[6-(nitromethyl)bicyclo[3.2.0]hept-6-yl]acetic acid

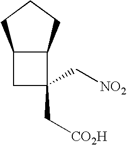

A solution of the nitro ester of preparation 31 (200 g; 828.9 mmol) in THF (1.0 L) was combined with 2M aq. NaOH (1.04 L; 2.08 mol) and stirred at ambient temperature for 18 hours. The biphasic mixture was diluted with toluene (500 mL) and the layers separated. The aqueous was adjusted to pH 1-3 with conc. aq. HCl and extracted with CH₂Cl₂ (1.0 L+600 mL). The combined product-containing CH₂Cl₂ layers were concentrated under reduced pressure to yield the title compound as an orange oil, which set to a solid (163.4 g).

¹H-NMR (400 MHz, CDCl₃): δ=4.80 (2H, m), 2.85 (1H, m), 2.60 (3H, m), 2.20 (1H, m), 1.85 (1H, m), 1.70 (2H, m), 1.6-1.4(4H, m).

### Preparation 33

### (1RS,5RS,6SR)-Spiri[bicyclo[3.2.0]heptane-6,3′-pyrrolidin]-5′-one

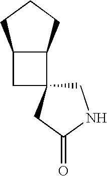

The nitroester of preparation 31 (13.0 g, 53.9 mmol) was shaken in methanol (125 ml) at 25° C. over Nickel sponge catalyst under an atmosphere of hydrogen gas at 345 kPa (50 p.s.i.). After 24 hrs the catalyst was removed by filtration through Arbocel and the solvent evaporated under reduced pressure. The residue was then chromatographed (SiO₂, ethyl acetate) to give the lactam (4.76 g).

¹H-NMR (400 MHz, CDCl₃): δ=5.86 (1H, br. s), 3.40 (2H, s), 2.79-2.70 (1H, m), 2.54-2.47 (1H, m), 2.32 (1H, d), 2.12 (1H, t), 2.03 (1H, d), 1.86-1.60 (3H, m), 1.57-1.38 (4H, m). Microanalysis: Found: C, 72.48; H, 9.15; N, 8.43. C₁₀H₁₅NO requires C, 72.69; H, 9.15; N, 8.48%. [α]_{D}-28.4° (25° C.)

### Pharmaceutical Composition Examples

In the following Examples, the active compound can be any compound of formula I-XXV and/or a pharmaceutically acceptable salt, solvate, or physiologically functional derivative thereof.

### (i) Tablet Compositions

The following compositions A and B can be prepared by wet granulation of ingredients (a) to (c) and (a) to (d) with a solution of povidone, followed by addition of the magnesium stearate and compression.

| Composition A | mg/tablet | mg/tablet |
|---|---|---|
| (a) Active ingredient | 250 | 250 |
| (b) Lactose B.P. | 210 | 26 |
| (c) Sodium Starch Glycollate | 20 | 12 |
| (d) Povidone B.P. | 15 | 9 |
| (e) Magnesium Stearate | 5 | 3 |
| | 500 | 300 |

| Composition B | mg/tablet | mg/tablet |
|---|---|---|
| (a) Active ingredient | 250 | 250 |
| (b) Lactose 150 | 150 | — |
| (c) Avicel PH 101 | 60 | 26 |
| (d) Sodium Starch Glycollate | 20 | 12 |
| (e) Povidone B.P. | 15 | 9 |
| (f) Magnesium Stearate | 5 | 3 |
| | 500 | 300 |

| Composition C | mg/tablet |
|---|---|
| Active ingredient | 100 |
| Lactose | 200 |
| Starch | 50 |
| Povidone | 5 |
| Magnesium Stearate | 4 |
| | 359 |

The following compositions D and E can be prepared by direct compression of the admixed ingredients. The lactose used in formulation E is of the direct compression type.

| Composition D | mg/tablet |
|---|---|
| Active ingredient | 250 |
| Magnesium Stearate | 4 |
| Pregelatinised Starch NF15 | 146 |
| | 400 |

| Composition E | mg/tablet |
|---|---|
| Active ingredient | 250 |
| Magnesium Stearate | 5 |
| Lactose | 145 |
| Avicel | 100 |
| | 500 |

| Composition F (Controlled release composition) | mg/tablet |
|---|---|
| (a) Active ingredient | 500 |
| (b) Hydroxypropylmethylcellulose | 112 |
| (Methocel K4M Premium) | |
| (c) Lactose B.P. | 53 |
| (d) Povidone B.P.C. | 28 |
| (e) Magnesium Stearate | 7 |
| | 700 |

The composition can be prepared by wet granulation of ingredients (a) to (c) with a solution of povidone, followed by addition of the magnesium stearate and compression.

### Composition G (Enteric-Coated Tablet)

Enteric-coated tablets of Composition C can be prepared by coating the tablets with 25 mg/tablet of an enteric polymer such as cellulose acetate phthalate, polyvinylacetate phthalate, hydroxypropylmethyl-cellulose phthalate, or anionic polymers of methacrylic acid and methacrylic acid methyl ester (Eudragit L). Except for Eudragit L, these polymers should also include 10% (by weight of the quantity of polymer used) of a plasticizer to prevent membrane cracking during application or on storage. Suitable plasticizers include diethyl phthalate, tributyl citrate and triacetin.

### Composition H (Enteric-Coated Controlled Release Tablet)

Enteric-coated tablets of Composition F can be prepared by coating the tablets with 50 mg/tablet of an enteric polymer such as cellulose acetate phthalate, polyvinylacetate phthalate, hydroxypropylmethyl-cellulose phthalate, or anionic polymers of methacrylic acid and methacrylic acid methyl ester (Eudgragit L). Except for Eudgragit L, these polymers should also include 10% (by weight of the quantity of polymer used) of a plasticizer to prevent membrane cracking during application or on storage. Suitable plasticizers include diethyl phthalate, tributyl citrate and triacetin.

### (ii) Capsule Compositions

### Composition A

Capsules can be prepared by admixing the ingredients of Composition D above and filling two-part hard gelatin capsules with the resulting mixture. Composition B (infra) may be prepared in a similar manner.

| Composition B | mg/capsule |
|---|---|
| (a) Active ingredient | 250 |
| (b) Lactose B.P. | 143 |
| (c) Sodium Starch Glycollate | 25 |
| (d) Magnesium Stearate | 2 |
| | 420 |

| Composition C | mg/capsule |
|---|---|
| (a) Active ingredient | 250 |
| (b) Macrogol 4000 BP | 350 |
| | 600 |

Capsules can be prepared by melting the Macrogol 4000 BP, dispersing the active ingredient in the melt and filling two-part hard gelatin capsules therewith.

| Composition D | mg/capsule |
|---|---|
| Active ingredient | 250 |
| Lecithin | 100 |
| Arachis Oil | 100 |
| | 450 |

Capsules can be prepared by dispersing the active ingredient in the lecithin and arachis oil and filling soft, elastic gelatin capsules with the dispersion.

| Composition E (Controlled release capsule) | mg/capsule |
|---|---|
| (a) Active ingredient | 250 |
| (b) Microcrystalline Cellulose | 125 |
| (c) Lactose BP | 125 |
| (d) Ethyl Cellulose | 13 |
| | 513 |

The controlled release capsule formulation can be prepared by extruding mixed ingredients (a) to (c) using an extruder, then spheronising and drying the extrudate. The dried pellets are coated with a release controlling membrane (d) and filled into two-part, hard gelatin capsules.

| Composition F (Enteric capsule) | mg/capsule |
|---|---|
| (a) Active ingredient | 250 |
| (b) Microcrystalline Cellulose | 125 |
| (c) Lactose BP | 125 |
| (d) Cellulose Acetate Phthalate | 50 |
| (e) Diethyl Phthalat | 5 |
| | 555 |

The enteric capsule composition can be prepared by extruding mixed ingredients (a) to (c) using an extruder, then spheronising and drying the extrudate. The dried pellets are coated with an enteric membrane (d) containing a plasticizer (e) and filled into two-part, hard gelatin capsules.

### Composition G (Enteric-Coated Controlled Release Capsule)

Enteric capsules of Composition E can be prepared by coating the controlled-release pellets with 50 mg/capsule of an enteric polymer such as cellulose acetate phthalate, polyvinylacetate phthalate, hydroxypropylmethylcellulose phthalate, or anionic polymers of methacrylic acid and methacrylic acid methyl ester (Eudragit L). Except for Eudragit L, these polymers should also include 10% (by weight of the quantity of polymer used) or a plasticizer to prevent membrane cracking during application or on storage. Suitable plasticizers include diethyl phthalate, tributyl citrate and triacetin.

| (iii) Intravenous injection composition | | |
|---|---|---|
| Active ingredient | 0.200 | g |
| Sterile, pyrogen-free phosphate buffer (pH 9.0) to | 10 | ml |

The active ingredient is dissolved in most of the phosphate buffer at 35-40° C., then made up to volume and filtered through a sterile micropore filter into sterile 10 ml glass vials (Type 1) which are sealed with sterile closures and overseals.

| (iv) Intramuscular injection composition | | |
|---|---|---|
| Active ingredient | 0.20 | g |
| Benzyl Alcohol | 0.10 | g |
| Glycofurol 75 | 1.45 | g |
| Water for Injection q.s. to | 3.00 | ml |

The active ingredient is dissolved in the glycofurol. The benzyl alcohol is then added and dissolved, and water added to 3 ml. The mixture is then filtered through a sterile micropore filter and sealed in sterile 3 ml glass vials (Type 1).

| (v) Syrup composition | | |
|---|---|---|
| Active ingredient | 0.25 | g |
| Sorbitol Solution | 1.50 | g |
| Glycerol | 1.00 | g |
| Sodium Benzoate | 0.005 | g |
| Flavour | 0.0125 | ml |
| Purified Water q.s. to | 5.0 | ml |

The sodium benzoate is dissolved in a portion of the purified water and the sorbitol solution added. The active ingredient is added and dissolved. The resulting solution is mixed with the glycerol and then made up to the required volume with the purified water.

| (vi) Suppository composition | mg/suppository |
|---|---|
| Active ingredient | 250 |
| Hard Fat, BP (Witepsol H15 - Dynamit NoBel) | 1770 |
| | 2020 |

One-fifth of the Witepsol H15 is melted in a steam-jacketed pan at 45° C. maximum. The active ingredient is sifted through a 2001 m sieve and added to the molten base with mixing, using a Silverson fitted with a cutting head, until a smooth dispersion is achieved. Maintaining the mixture at 45° C., the remaining Witepsol H15 is added to the suspension which is stirred to ensure a homogenous mix. The entire suspension is then passed through a 2501 m stainless steel screen and, with continuous stirring, allowed to cool to 40° C. At a temperature of 38-40° C., 2.02 g aliquots of the mixture are filled into suitable plastic moulds and the suppositories allowed to cool to room temperature.

| (vii) Pessary composition | mg/pessary |
|---|---|
| Active ingredient (631 m) | 250 |
| Anhydrous Dextrose | 380 |
| Potato Starch | 363 |
| Magnesium Stearate | 7 |
| | 1000 |

The above ingredients are mixed directly and pessaries prepared by compression of the resulting mixture.

| (viii) Transdermal composition | | |
|---|---|---|
| Active ingredient | 200 | mg |
| Alcohol USP | 0.1 | ml |
| Hydroxyethyl cellulose | | |

The active ingredient and alcohol USP are gelled with hydroxyethyl cellulose and packed in a transdermal device with a surface area of 10 cm².

### Biological Data

The compound of examples 1 and 4 were tested in the radioligand binding assay described herein and were found to have binding affinities of 46.8 and 600 nM respectively.

1. A method for treating fibrmyalgia in a mammal comprising administering to the mammal a therapeutically effective amount of a compound of any of the formulae (I)-(XXV): 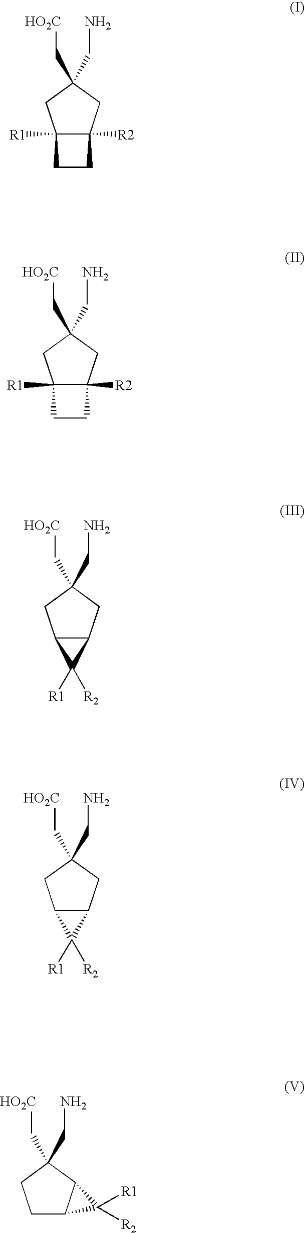 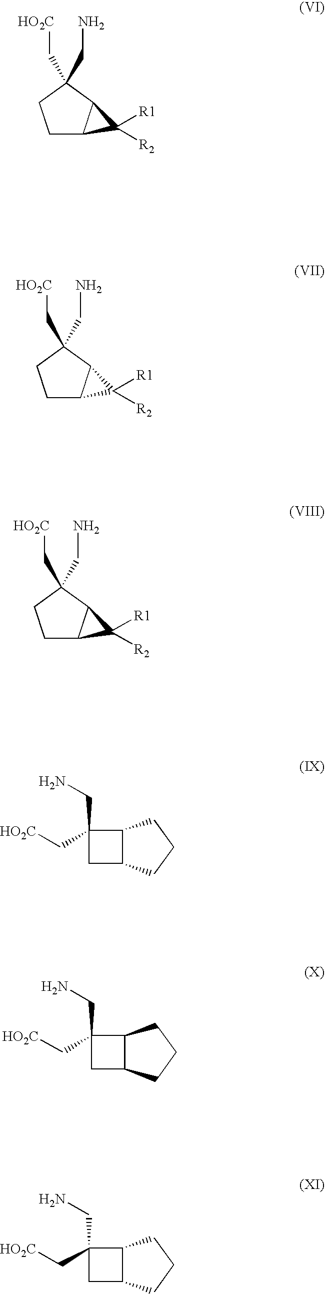 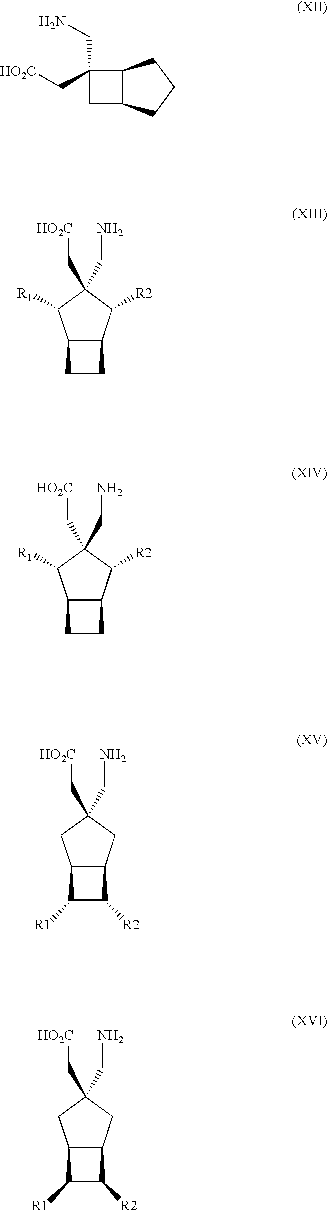 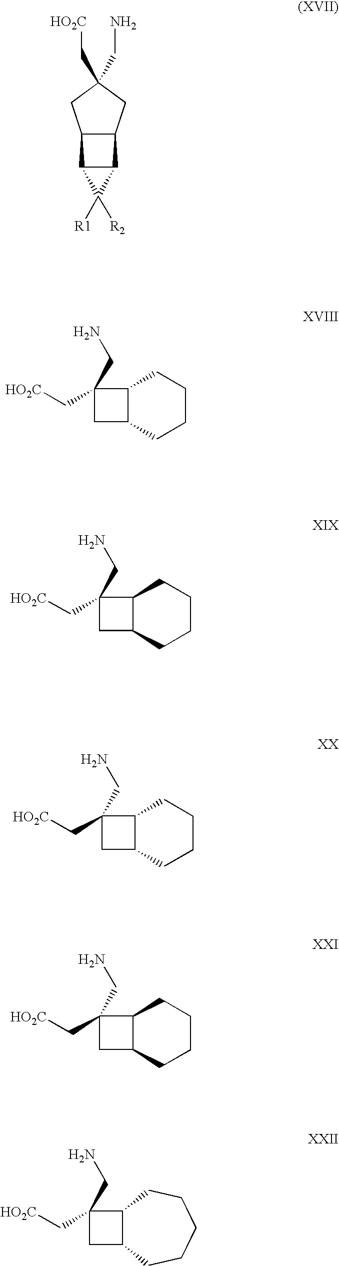 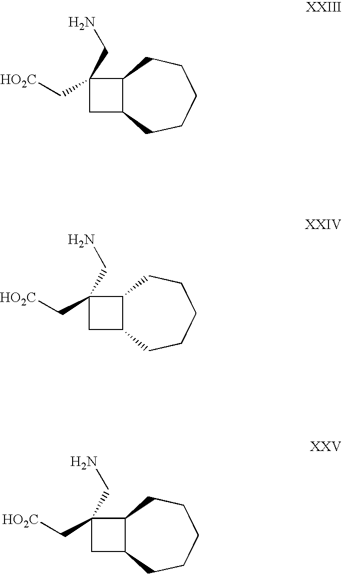
wherein R¹ and R² are each independently selected from hydrogen, straight or branched alkyl of 1-6 carbon atoms, cycloalkyl of from 3-6 carbon atoms, phenyl and benzyl, subject to the proviso that except in the case of a tricyclooctane compound of formula (XVII) R¹ and R² are not simultaneously hydrogen; or a pharmaceutically acceptable salt or solvate thereof; or a prodrug thereof.

2. A method according to claim 1, wherein R¹ and R² are both hydrogen or methyl.

3. A method according to claim 1, wherein the compound is selected from:
((1R,5S)-3-Aminomethyl-1,5-dimethyl-bicyclo[3.2.0]hept-3-yl)-acetic acid;
((1S,5R)-3-Aminomethyl-1,5-dimethyl-bicyclo[3.2.0]hept-3-yl)-acetic acid;
((1R,5S)-3-Aminomethyl-6,6-dimethyl-bicyclo[3.1.0]hex-3-yl)-acetic acid;
((1S,5R)-3-Aminomethyl-6,6-dimethyl-bicyclo[3.1.0]hex-3-yl)-acetic acid;
((1S,2S,5R)-2-Aminomethyl-6,6-dimethyl-bicyclo[3.1.0]hex-2-yl)-acetic acid;
((1R,2S,5S)-2-Aminomethyl-6,6-dimethyl-bicyclo[3.1.0]hex-2-yl)-acetic acid;
((1S,2R,5R)-2-Aminomethyl-6,6-dimethyl-bicyclo[3.1.0]hex-2-yl)-acetic acid;
((1R,2R,5S)-2-Aminomethyl-6,6-dimethyl-bicyclo[3.1.0]hex-2-yl)-acetic acid;
((1R,5R,6S)-6-Aminomethyl-bicyclo[3.2.0]hept-6-yl)-acetic acid;
((1S,5 S,6S)-6-Aminomethyl-bicyclo[3.2.0]hept-6-yl)-acetic acid;
((1R,5R,6R)-6-Aminomethyl-bicyclo[3.2.0]hept-6-yl)-acetic acid;
((1S,5 S,6R)-6-Aminomethyl-bicyclo[3.2.0]hept-6-yl)-acetic acid;
cis-((1S,2R,4S,5R)-3-Aminomethyl-2,4-dimethyl-bicyclo[3.2.0]hept-3-yl)acetic acid;
trans-((1S,2R,4S,5R)-3-Aminomethyl-2,4-dimethyl-bicyclo[3.2.0]hept-3-yl)acetic acid;
((1S,5R,6S,7R)-3-Aminomethyl-6,7-dimethyl-bicycl[3.2.0]hept-3-yl)acetic acid;
((1S,5R,6R,7S)-3-Aminomethyl-6,7-dimethyl-bicyclo[3.2.0]hept-3-yl)acetic acid;
((1R,2S,5S)-7-Aminomethyl-3,3-dimethyl-tricyclo[3.3.0.0]oct-7-y)-acetic acid:
((1R,6R,7S)-7-Aminomethyl-bicyclo[4.2.0]oct-7-yl)-acetic acid;
((1S,6S,7S)-7-Aminomethyl-bicyclo[4.2.0]oct-7-yl)-acetic acid;
((1R,6R,7R)-7-Aminomethyl-bicyclo[4.2.0]oct-7-yl)-acetic acid;
((1S,6S,7R)-7-Aminomethyl-bicyclo[4.2.0]oct-7-yl)-acetic acid;
((1R,7R,8S)-8-Aminomethyl-bicyclo[5.2.0]non-8-yl)-acetic acid;
((1S,7S,8S)-8-Aminomethyl-bicyclo[5.2.0]non-8-yl)-acetic acid;
((1R,7R,8R)-8-Aminomethyl-bicyclo[5.2.0]non-8-yl)-acetic acid; and
((1S,7S,8R)-8-Aminomethyl-bicyclo[5.2.0]non-8-yl)-acetic acid.

4. A method according to claim 1, wherein the compound is selected from:
[(1R,5R,6S)-6-(Aminomethyl)bicyclo[3.2.0]hept-6-yl]acetic acid;
[(1S,5S,6R)-6-(Aminomethyl)bicyclo[3.2.0]hept-6-yl]acetic acid;
[(1RS,5RS,6RS)-6-(Aminomethyl)bicyclo[3.2.0]hept-6-yl]acetic acid;
[(1RS,6RS,7SR)-7-(Aminomethyl)bicyclo[4.2.0]oct-7-yl]acetic acid; and
[(1RS,6RS,7RS)-7-(Aminomethyl)bicyclo[4.2.0]oct-7-yl]acetic acid.

5. A method according to claim 1, wherein the compound is [(1R,5R,6S)-6(Aminomethyl)bicyclo[3.2.0]hept-6-(Aminomethyl)bicyclo[3.2.0]hept-6-yl]acetic acid.

6. A pharmaceutical composition comprising a therapeutically effective amount of a compound of formula (I)-(XXV) according to claim 1 and a pharmaceutically acceptable carrier.
